(19) United States
(12) Patent Application Publication
(10) Pub. No.: US 2026/0209314 A1
(43) Pub. Date: Jul. 23, 2026

(54) MODIFIED POLYPEPTIDES AND METHOD FOR PREPARING SAME

(71) Applicant: B. G. NEGEV TECHNOLOGIES AND APPLICATIONS LTD., AT BEN-GURION UNIVERSITY, Beer Sheva (IL)

(72) Inventors: Lital ALFONTA, Beer Sheva (IL); Smadar COHEN, Beer Sheva (IL); Mor PASI-DEUTSCHMAN, Beer Sheva (IL)

(73) Assignee: B. G. NEGEV TECHNOLOGIES AND APPLICATIONS LTD., AT BEN-GURION UNIVERSITY, Beer Sheva (IL)

(21) Appl. No.: 19/157,887

(22) PCT Filed: Feb. 21, 2024
(86) PCT No.: PCT/IL2024/050197

§ 371 Date: Aug. 19, 2025

Related U.S. Application Data
(60) Provisional application No. 63/447,051, filed on Feb. 21, 2023.

(51) Int. Cl. C07K 14/78; C12N 15/70

(52) U.S. Cl. CPC C07K 14/78; C12N 15/70

(57) ABSTRACT

Disclosed herein is a polypeptide including an amino acid residue substituted by 1-3,4-dihydroxyphenylalanine (L-DOPA) or a functional analog thereof. Further provided are compositions comprising the disclosed polypeptide and methods for producing the same.

### CROSS-REFERENCE TO RELATED-APPLICATIONS

This application claims the benefit of priority of U.S. Provisional Patent Application No. 63/447,051, titled “MODIFIED POLYPEPTIDES AND METHOD FOR PREPARING SAME”, filed 21 Feb. 2023, the contents of which are incorporated herein by reference in their entirety.

### REFERENCE TO AN ELECTRONIC SEQUENCE LISTING

The contents of the electronic sequence listing (BGU-P-0110-PCT.xml; size: 4,859 bytes; and date of creation: Jan. 21, 2024) is herein incorporated by reference in its entirety.

### FIELD OF THE INVENTION

The present invention, in some embodiments, is in the field of molecular biology and biochemistry, and is specifically directed to modified polypeptides, and methods for preparing same.

### BACKGROUND

Collagen is an important structural protein in the extracellular matrix (ECM), which provides physical and biological support for cells. In tissue engineering, Collagen is utilized to create scaffolds, due to its biocompatibility and biodegradability in addition to the cell support properties. Usually, Collagen is extracted from animal tissues, in an expansive process. Although the process involves purification stages, there is a risk for pathogen transition to future applications.

In recent years, research has focused on finding other solutions for recombinant collagen production in other systems, bacterial or plant. It is possible to produce engineered collagen mimicking polypeptides in Escherichia coli that self-assemble to form collagen fibrils, but these polypeptides do not possess post-translational modifications.

There is still a great need for collagen mimicking polypeptides with increased adhesion, such as for use as bio-adhesive biomaterial, for example, in medical applications.

### SUMMARY

According to a first aspect, there is provided a polypeptide comprising at least 80% homology to an amino acid sequence comprising: MGSGYGSGSWCGTTPGSWCGT-[GPP]ₙ-GRDGSPGGSGYGSG, wherein an amino acid residue located C-terminally to the [GPP]ₙ repeat is substituted by 1-3,4-dihydroxyphenylalanine (L-DOPA) or a functional analog thereof, and wherein n is the number of repetitions of the [GPP] repeat.

According to another aspect, there is provided a polynucleotide comprising a nucleic acid sequence encoding the polypeptide of the invention.

According to another aspect, there is provided an artificial vector comprising the polynucleotide of the invention.

According to another aspect, there is provided a cell comprising: (a) the polypeptide of the invention; (b) the polynucleotide of the invention; (c) the artificial vector of the invention; or (d) any combination of (a) to (c).

According to another aspect, there is provided a composition comprising any one of: (a) the polypeptide of the invention; (b) the polynucleotide of the invention; (c) the artificial vector of the invention; (d) the cell of the invention; and (e) any combination of (a) to (d), and an acceptable carrier.

According to another aspect, there is provided a method for producing the polypeptide of the invention, comprising the steps: (a) providing a transformed bacterial cell comprising the artificial vector of the invention; and (b) culturing the transformed cell from step (a), wherein the culturing comprises contacting the transformed bacterial cell with a sufficient amount of L-DOPA, thereby producing the polypeptide of the invention.

According to another aspect, there is provided a method for producing a polypeptide comprising at least 80% homology to an amino acid sequence comprising MGSGX₁GSGSWCGTTPGSWCGT-[GPP]ₙ-GRDGSPGGSGX₂GSG, wherein n is the number of repetitions of the [GPP] repeat, and wherein any one of X₁ and X₂ is Tyrosine or L-DOPA, and: (i) when X₁ is Tyrosine X₂ is L-DOPA, and vice versa; or (ii) both X₁ and X₂ are L-DOPA, the method comprising contacting a polypeptide comprising at least 80% homology to the amino acid sequence: MGSGYGSGSWCGTTPGSWCGT-[GPP]ₙ-GRDGSPGGSGYGSG with an effective amount of a tyrosinase enzyme, or a functional analog thereof.

According to another aspect, there is provided a composition comprising a plurality of molecules of the polypeptide produced according to the method of the invention, and an acceptable carrier.

In some embodiments, the amino acid residue located C-terminally to the [GPP]ₙ repeat is a tyrosine residue.

In some embodiments, the polypeptide comprises the amino acid sequence: MGSGYGSGSWCGTTPGSWCGT-[GPP]ₙ-GRDGSPGGSGXGSG, wherein X is L-DOPA.

In some embodiments, the polypeptide comprises the amino acid sequence: MGSGX₁GSGSWCGTTPGSWCGT-[GPP]ₙ-GRDGSPGGSGX₂GSG, wherein n is the number of repetitions of the [GPP] repeat, wherein any one of X₁ and X₂ is Tyrosine or L-DOPA

In some embodiments, (i) when X₁ is Tyrosine X₂ is L-DOPA, and vice versa; or (ii) both X₁ and X₂ are L-DOPA.

In some embodiments, n represents an integer ranging from 1 to 50.

In some embodiments, n equals 12.

In some embodiments, the polypeptide comprises the amino acid sequence:

MGSGX₁GSGSWCGTTPGSWCGTGPPGPPGPPGPPGPPGPPGPPGPPGPPGPPGPPG PPGRDGSPGGSGX₂GSG (SEQ ID NO: 1), wherein: (i) when X₁ is Tyrosine X₂ is L-DOPA, or vice versa; or (ii) both X₁ and X₂ are L-DOPA.

In some embodiments, the polypeptide comprises the amino acid sequence: MGSGX₁GSGSWCGTTPGSWCGTGPPGPPGPPGPPGPPGPPGPPGPPGPPGPPGPPG PPGRDGSPGGSGX₂GSG (SEQ ID NO: 4), wherein X₁ and X₂ are L-DOPA.

In some embodiments, the polypeptide is in the form of a fiber.

In some embodiments, the fiber is characterized by a length ranging from 5 nm to 1.5 μm.

In some embodiments, the fiber is characterized by a diameter ranging from 0.1 nm to 10 μm.

In some embodiments, the polypeptide is characterized by a porosity ranging from 30% to 90%.

In some embodiments, the polypeptide is characterized by a tensile strength ranging from 10 MPa to 500 MPa.

In some embodiments, the polypeptide is characterized by a young's modulus ranging from 1 KPa to 15 KPa. In some embodiments, the polypeptide is characterized by young's modulus ranging from 5 KPa to 20 KPa.

In some embodiments, the polypeptide further comprises a cross-linking modification.

In some embodiments, the polynucleotide comprises the nucleic acid sequence:

(SEQ ID NO: 2)
ATGGGTAGCGGTTATGGCAGCGGTAGCTGGTGCGGCACCACCCCGGGCA
GCTGGTGCGGTACCGGTCCGCCGGGTCCGCCGGGTCCGCCGGGCCCACC
GGGTCCGCCGGGTCCGCCAGGTCCACCGGGCCCGCCGGGCCCGCCAGGT
CCGCCGGGTCCACCGGGTCCGCCGGGTCGTGATGGTAGCCCGGGTGGTA
GTGGATAGGGAAGTGGA.

In some embodiments, the artificial vector is an expression vector or a plasmid.

In some embodiments, the cell is a prokaryotic cell.

In some embodiments, the cell of is an Escherichia coli cell.

In some embodiments, the composition is in the form of: a sponge, a hydrogel, a hollow fiber tubing, a film, an ink, a patch, or a membrane.

In some embodiments, the composition is a porous composition.

In some embodiments, the porous composition comprises pores with an average size ranging between 50 μm to 500 μm.

In some embodiments, the composition is for use in any one of: wound healing, three-dimensional (3D) cell culturing, nerve regeneration, tissue regeneration, bone repair, skin graft, cosmetics, 3D printing, cardiac patches, and any combination thereof.

In some embodiments, the method further comprises a step preceding step (a), comprising transforming or transfecting the artificial vector into a bacterial cell, thereby providing the transformed bacterial cell.

In some embodiments, the method further comprises a step (c), comprising isolating, purifying, or both, the produced polypeptide.

In some embodiments, the transformed cell further comprises a transfer RNA (tRNA) synthase capable of ligating L-DOPA to a tRNA molecule.

In some embodiments, the tRNA synthase is a modified tyrosyl-tRNA synthase.

In some embodiments, the anti-codon of the tRNA molecule comprises a nucleic acid sequence of CUA.

In some embodiments, the method further comprises a step preceding step (a) comprising transforming or transfecting the bacterial cell with a polynucleotide comprising any one of: a nucleic acid sequence of the tRNA molecule, a nucleic acid sequence encoding the tRNA synthase, and both.

In some embodiments, the polynucleotide comprises a plurality of polynucleotide types.

In some embodiments, a first type of the plurality of polynucleotide types comprises a nucleic acid sequence of the tRNA molecule, and a second type of the plurality of polynucleotide types encodes the tRNA synthase.

In some embodiments, at least 80% of the plurality of molecules of the polypeptide comprise a L-DOPA.

In some embodiments, at least 95% of the plurality of molecules of the polypeptide comprise a L-DOPA.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include micrographs of anti-His western-blot of His-MBP-GPPn expressed in BL21 cells, after cell lysis samples were separated to supernatant(S) and pellet (P) for n=12 (1A; twelve GPP repeats), and n=50 (1B; fifty GPP repeats).

FIG. 2 includes a micrograph of a Coomassie blue staining of His-MBP-GPP12 cleavage by the TEV protease.

FIG. 3 includes a fluorescent micrograph of a gel imaging of His-MBP-GPP12 wherein tyrosine on position 5 (Y5) was substituted. (+) Presence or (−) absence of 1 mM Propargyl-L-lysine (PrK) after a click reaction to link TAMRA-Az.

FIG. 4 includes a micrograph of anti-chitin binding domain (CBD) western blot of GPP12-Mxe-CBD expressed in BL21 cells; after cell lysis samples were separated into supernatant(S) and pellet (P). Mxe represents an Intein sequence taken from Mycobacterium xenopi that can be cleaved under reducing conditions.

FIG. 5 includes a graph showing matrix-assisted laser desorption/ionization time of flight (MALDI-TOF) spectrum of purified GPP12.

FIG. 6 includes a micrograph of anti-CBD western blot of GPP12-Mxe-CBD expressed in BL21 cell, wherein tyrosine on position 68 (Y68) was substituted with L-DOPA. (+) Presence (−) or absence of 2 mM of L-DOPA in the medium.

FIG. 7 includes a graph showing cyclic voltammograms of GPP12 and GPP12 wherein tyrosine on position 68 was substituted with L-DOPA (Y68[L-DOPA]).

FIG. 8 includes a micrograph of anti-His tag western blot of GFP-His expressed in DH5a cells, wherein tyrosine 35 (Y35) was substituted with L-DOPA. (+) Presence or (−) absence of 2 mM of L-DOPA in the medium.

FIG. 9 includes a micrograph showing the conjugation between TAMRA-amine and purified GFP Y35 with L-DOPA or Prk.

FIG. 10 includes a micrograph showing the conjugation between TAMRA-amine and GFP Y35 [L-DOPA]. (1) lysate of bacteria that were grown in the presence of 2 mM L-DOPA; (2) lysate of bacteria that were grown in the absence of L-DOPA; and (P) a purified and concentrated GFP Y35 [L-DOPA].

FIGS. 11A-11B include graphs of circular dichroism (CD) spectra of 0.1 mg/mL GPP12 in PBS (11A) and 0.01 mg/mL GPP12 (11B).

FIG. 12 includes transmission electron microscopy (TEM) micrographs of 0.2 mg/mL GPP12. Scale bar=0.5 μm (right), and 0.2 μm (left). The left micrograph represents an enlargement of the area framed in the right micrograph.

FIGS. 13Ai-13Dii include TEM micrographs of 3.8 mg/ml (PBS, pH 7.4) GPP12. (13Ai-13Aii) Control-Fresh GPP12 without any treatment; Scale bar=100 nm (13Ai), and 0.2 μm (13Aii). (13Bi-13Bv) GPP12 incubated at 37° C. for 7 days. Small and large fibrils (13Bi-13Biii) and fibrous network (13Biv-13Bv); Scale bar=0.2 μm (13Bi-13Bii, and 13Biv), 0.5 μm (13Biii) and 1 μm (13Bv). (13Ci-13Dii) Over-night (13Ci-13Cii) and 7 days incubation (13Di-13Dii) of GPP12 with 0.1% H₂O₂ at 37° C. Only small fibrils were observed, regardless of the incubation time.

FIGS. 14A-14C include micrographs and a vertical bar graph. (14A-14B) Micrographs showing 1% bovine Achilles tendon collagen sponges: SEM images of the porous structure scaffolds prepared without cross-linking (14A), and scaffolds prepared with chemical 1-ethyl-3-(3-dimethylaminopropyl) carbodiimide N-hydroxysuccinimide (EDC-NHS) cross-linking (14B). (14C) A graph of modulus Young measurement with and without cross-linking, n=12.

FIGS. 15A-15D include bright-field and fluorescent micrographs of: (15A) GPP12; (15B) GPP12 Y5 [L-DOPA]; (15C) GPP12 Y68[L-DOPA]; and (15D) collagen Type I (rat tail).

FIGS. 16A-16C include a graph and micrographs showing that oxidation can effectively control the self-assembly of the peptides of the invention. (16A) Turbidity assay at 313 nm of 0.5-1 mg/mL GPP12 and GPP12Y68[L-DOPA] with and without 0.5 mM NaIO₄, data presented as means of 3 repetitions. (16B-16C) CryoTEM images: (16B) 3 mg/mL GPP12 and GPP12Y68[L-DOPA] incubated over-night with and without 0.5 mM NaIO₄, scale bar 500 nm. (16C) 0.5 mg/mL GPP12Y68[L-DOPA] incubated over-night with and without NaIO₄, scale bar 2 μm.

FIGS. 17A-17C include photographs, a vertical bar graph, and fluorescent micrographs showing that the peptides of the invention self-assemble into hydrogels. (17A) Peptide gelation assay, peptide solution at time 0 and after 30 minutes incubation at 4° C. and 25° C. (17B) HFGFP viability at day 1 and day 4 in the presence of 0-2 mM CuSO₄ and on GPP12Y68[L-DOPA]+2 mM CuSO₄ thin film. Two-way ANOVA, **** p<0.0001. (17C) Fluorescent images of HFGFP (expressing GFP in green) at day 4, 0 mM CuSO₄ as a control and on GPP12Y68[L-DOPA]+2 mM CuSO₄ thin film, scale bar 100 μm.

FIGS. 18A-18C include micrographs and graphs showing that the peptides of the invention self-assemble into hydrogels. (18A) Confocal images of peptides sheets after TAMRA amin staining: (i) z-stack max intensity projection of 0.3 mg/mL GPP12Y68[L-DOPA] and GPP12, scale bar 100 μm. (ii) z-stack side view of 0.3 mg/mL GPP12Y68[L-DOPA] sheets, scale bar 100 μm. (iii) average area of GPP12Y68[L-DOPA] sheets. (18B-18C) Mean viscosity of 0.5 mg/mL GPP12Y68[L-DOPA] with 0-2 mM NaIO₄ (18B) and 2% VLVG 0.75% CaGlu in combination with 0.5 mg/mL GPP12Y68[L-DOPA] and 0.5 NaIO₄ (18C) 5 repetitions per each sample.

FIGS. 19A-19D include a scheme, fluorescent micrographs and graphs showing the cell adhesion properties of the L-DOPA collagen scaffold. (19A) A non-limiting experimental setup. Cross-linked alginate (2% VLVG+0.75% Ca) was mixed with 0.5 mg/mL collagen-mimicking peptides (GPP12 or GPP12Y68[L-DOPA]). Subsequently, thin films were dried, and HFGFP cell were incubated on these films. (19B) Confocal images z-stack max intensity projection of cells after phalloidin (red) and DAPI (blue) staining for F-actin and nucleus, cells expressed GFP (green), scale bar 100 μm. (19C) HFGFP viability at day 4 relative to day 1. (19D) HFGFP cell's area and circularity.

FIGS. 20A-20B include a vertical bar graph and fluorescent micrographs showing that HFGFP cells successfully proliferate and spread on hydrogels made of oxidized GPP12Y68[L-DOPA]. HFGFP cells were cultured on sheets prepared from 0.5 mg/mL GPP12Y68[L-DOPA] with 0.1-2 mM NaIO₄ without and with DMEM was: (20A) viability at day 1 and day 4. (20B) Confocal images z-stack max intensity projection of cells at day 4 after phalloidin (red) and DAPI (blue) staining for F-actin and nucleus, expressed GFP (green), scale bar 200 μm.

FIG. 21 includes a fluorescent micrograph of HFGFP cells, expressing GFP (Green), on 1% LVG-GPP12Y68[L-DOPA]+0.14% Ca film at day 7. Scale bar 200 μm.

FIG. 22 includes a vertical bar graph showing scaffold porosity as a function of cross linker concentrations. Alginate-LVG.

FIGS. 23A-23D include micrographs of a scanning electron microscopy (SEM) showing 1% LVG (23A and 23C) and 1% LVG-GPP12Y68[L-DOPA] (23B and 23D) scaffolds cross-linked with 0.16% (23A-23B) or 0.17% (23C-23D) Calcium (Ca). Scale bar 300 μm.

FIGS. 24A-24D include micrographs of a scanning electron microscopy (SEM) showing 1% LVG-GPP12Y68[L-DOPA] scaffolds cross-linked with 0.05% (24A), 0.10% (24B), 0.12% (24C), or 0.14% (24D) Ca. Scale bar 300 μm.

FIGS. 25A-25H include micrographs of a scanning electron microscopy (SEM) showing 1% LVG-GPP12Y68[L-DOPA] (25A-25D) and 1% LVG (25E-25H) and scaffolds cross-linked with 0.12% Ca. Scale bar 300 μm. Top view (25A and 25E); Bottom view (25B and 25F); Vertical cut (25C and 25G); and horizontal cut (25D and 25H).

FIGS. 26A-26H include fluorescent micrographs of confocal microscopy analysis showing z-stack max intensity projection of HFGFP cells cultured for 1 day (25A and 25E), 4 days (25B and 25F), 7 days (25C and 25G) and 14 days (25D and 25H) on 1% LVG-GPP12Y68[L-DOPA] (25D-25H) and 1% LVG (25A-25D) scaffolds cross-linked with 0.16% Ca. Samples were stained with phalloidin (red), cells expressed GFP (green), scale bar 200 μm.

FIGS. 27A-27F include fluorescent micrographs of confocal microscopy analysis showing z-stack max intensity projection of HFGFP cells cultured for 1 day (27A and 27D), 4 days (27B and 27E), and 7 days (27C and 27F) on 1% LVG-GPP12Y68[L-DOPA] (27A-27C) and 1% LVG-GPP12 (27D-27F) scaffolds cross-linked with 0.12% Ca. Samples were stained with phalloidin (red) and NucBlue (blue), cells expressed GFP (green), scale bar 100 μm.

FIGS. 28A-28D include vertical bar graphs showing viability analysis of HFGFP cells cultured for 7 days on 1% LVG-GPP12Y68[L-DOPA] and 1% LVG-GPP12 scaffolds cross-linked with 0.12% Ca. (28A) A graph showing metabolic activity. (28B) A graph showing DNA quantification. (28C) A graph showing normalized cell viability calculated by the ratio between the metabolic activity and DNA quantification within a specific sample. (28D) A graph showing cell viability at Day 4 and day 7 relative to the viability at day 1. (n=3). * indicates significant difference at P<0.05.

### DETAILED DESCRIPTION

The present invention is directed to a polypeptide comprising a C-terminus amino acid residue substituted by 1-3,4-dihydroxyphenylalanine (L-DOPA) or a functional analog thereof.

The present invention is also directed to a method for producing or preparing the polypeptide described herein.

According to some embodiments, the present invention provides a polypeptide comprising an N-terminus, C-terminus, or both, amino acid residue substituted by 1-3,4-dihydroxyphenylalanine (L-DOPA) or a functional analog thereof. In some embodiments, the polypeptide is in the form of a fiber.

According to some embodiments, the present invention provides a polypeptide produced in a cell, comprising a C-terminus amino acid residue substituted by 1-3,4-dihydroxyphenylalanine (L-DOPA) or a functional analog thereof. In some embodiments, the polypeptide is in the form of a fiber.

In some embodiments, the present invention is based, in part, on the finding that an amino acid residue located in the N-terminus, C-terminus, or both, can be substituted in a site-specific and controlled manner, so as to increase fibrillation efficiency, adhesion properties, or both.

In some embodiments, the present invention is directed to a polypeptide comprising an amino acid sequence comprising: MGSGYGSGSWCGTTPGSWCGT-[GPP]ₙ-GRDGSPGGSGYGSG, wherein an amino acid residue located N-terminally, C-terminally, or both, to the [GPP]ₙ repeat is substituted by L-DOPA or a functional analog thereof, and wherein n is the number of repetitions of the [GPP] repeat. In some embodiments, n is 12.

In some embodiments, the polypeptide comprises the amino acid sequence: MGSGYGSGSWCGTTPGSWCGT-[GPP]ₙ-GRDGSPGGSGYGSG, wherein n is 12. In some embodiments, when n is 12, the tyrosine (Y) residue located N-terminally to the [GPP]₁₂ repeat is at position 5 (Y5), and the tyrosine residue located C-terminally to the [GPP]₁₂ repeat is at position 68 (Y68).

The present invention is based, in part, on the surprising finding that a polypeptide comprising an amino acid residue located in the N-terminus, C-terminus substituted, or both, by L-DOPA, exhibited increased adhesion properties without losing its tensile strength. In some embodiments, a polypeptide wherein the Y68 residue was substituted by L-DOPA exhibited increased adhesion properties without losing its tensile strength.

Surprisingly, the Y68[L-DOPA] modified polypeptide described herein, was found to exhibit increased adhesion properties compared to a polypeptide wherein a tyrosine residue in position 5 (Y5) was substituted by L-DOPA.

In some embodiments, the polypeptide of the invention is a collagen-mimicking polypeptide. In some embodiments, the polypeptide is a collagen-like polypeptide. In some embodiments, the polypeptide is a fibrous collagen polypeptide.

### Polypeptides

According to some embodiments, there is provided a polypeptide comprising at least 80% homology to an amino acid sequence comprising: MGSGYGSGSWCGTTPGSWCGT-[GPP]ₙ-GRDGSPGGSGYGSG, wherein an amino acid residue located C-terminally to the [GPP]ₙ repeat is substituted by L-DOPA, or a functional analog thereof, and wherein n is the number of repetitions of the [GPP] repeat.

In some embodiments, the polypeptide comprises at least 85%, at least 90%, at least 95%, at least 98%, or at least 99% homology or identity to the amino acid sequence: MGSGYGSGSWCGTTPGSWCGT-[GPP]ₙ-GRDGSPGGSGYGSG, or any value and range therebetween, wherein an amino acid residue located C-terminally to the [GPP]ₙ repeat is substituted by L-DOPA, or a functional analog thereof, and wherein n is the number of repetitions of the [GPP] repeat. Each possibility represents a separate embodiment of the invention.

In some embodiments, the amino acid residue located C-terminally to the [GPP]ₙ repeat is a tyrosine residue. In some embodiments, the polypeptide comprises the amino acid sequence: MGSGX₁GSGSWCGTTPGSWCGT-[GPP]ₙ-GRDGSPGGSGX₂GSG, wherein X₁ and/or X₂ are Tyrosine or L-DOPA. In some embodiments, when X₁ is Tyrosine X₂ is L-DOPA, or vice versa. In some embodiments, both X₁ and X₂ are L-DOPA.

In some embodiments, n represents any integer ranging from 1 to 50, 1 to 40, 1 to 30, 1 to 20, 1 to 15, 2 to 50, 2 to 40, 2 to 30, 2 to 20, 2 to 15, 2 to 50, 2 to 40, 2 to 30, 2 to 20, 2 to 15, 5 to 50, 5 to 40, 5 to 30, 5 to 20, 5 to 15, 10 to 50, 10 to 40, 10 to 30, 10 to 20, or 10 to 15, including any range therebetween. Each possibility represents a separate embodiment of the invention.

In some embodiments, n equals 12.

In some embodiments, the polypeptide comprises the amino acid sequence: MGSGX₁GSGSWCGTTPGSWCGTGPPGPPGPPGPPGPPGPPGPPGPPGPPGPPGPPG PPGRDGSPGGSGX₂GSG (SEQ ID NO: 1), wherein any one of X₁ and X₂ are Tyrosine or L-DOPA. In some embodiments, when X₁ is Tyrosine X₂ is L-DOPA, or vice versa. In some embodiments, when X₁ is L-DOPA X₂ is Tyrosine, or vice versa.

In some embodiments, the polypeptide comprises the amino acid sequence: MGSGX₁GSGSWCGTTPGSWCGTGPPGPPGPPGPPGPPGPPGPPGPPGPPGPPGPPG PPGRDGSPGGSGX₂GSG (SEQ ID NO: 4), wherein X₁ and X₂ are L-DOPA.

In some embodiments, the polypeptide is in the form of a fibril. In some embodiments, the polypeptide is in the form of a fiber.

As used herein, the terms “polypeptide”, “peptide” and “protein” are used interchangeably to refer to two or more amino acids linked together. The terms “polypeptide”, “peptide”, “protein”, and “amino acid sequence” as used herein refer to any compound comprising naturally occurring or synthetic amino acid polymers or amino acid-like molecules including but not limited to compounds comprising amino and/or imino molecules. No particular size is implied by use of the term “peptide”, “oligopeptide”, “polypeptide”, or “protein”. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, multiple antigenic peptide (MAP) forms, linearly linked peptides), cyclized, branched molecules and the like, are included within the definition.

The term “analog” as used herein, refers to a polypeptide that is similar, but not identical, to the polypeptide of the invention that still includes L-DOPA incorporated therewith, capable of self-assemble, fibrillate, adhere to cells or vice versa, or any combination thereof. An analog may have deletions or mutations that result in an amino acids sequence that is different than the amino acid sequence of the polypeptide of the invention. It should be understood that all functional analogs of the polypeptide of the invention would still be capable of self-assembling, fibrillate, adhering to cells or vice versa, or any combination thereof. Further, a functional analog may be analogous to a fragment of the polypeptide of the invention, however, in such a case the fragment must comprise at least 50 consecutive amino acids of the polypeptide of the invention.

In some embodiments, a functional analog comprises a functional analog of L-DOPA. In some embodiments, a functional analog of L-DOPA comprises any compound capable of cross linking, binding, or the like, such as may be induced by oxidation.

In some embodiments, an analog to the polypeptide of the invention comprises an amino acid sequence with at least 60%, at least 65%, at least 70%, at least 75%, at least 80%, at least 85%, at least 90%, at least 95%, at least 99% homology to the amino acid sequence presented in SEQ ID NO: 1.

In some embodiments, an analog to the polypeptide of the invention comprises an amino acid sequence with at least 60%, at least 65%, at least 70%, at least 75%, at least 80%, at least 85%, at least 90%, at least 95%, at least 99% homology to the amino acid sequence presented in SEQ ID NO: 4.

As used herein, the term “analog” includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

As used herein, the term “homology” refers to the percent identity between two aligned sequences, e.g., either polynucleotides or polypeptide. In some embodiments, two sequences, e.g., nucleic acids, or polypeptides are “substantially homologous” to each other when their sequences exhibit at least 50%, at least 75%, at least 85%, at least 90%, at least 95%, or at least 98% identity over a defined length of the nucleic acids, or polypeptides. Each possibility represents a separate embodiment of the invention.

As used herein, the terms “homologous” and “substantially homologous” refers to sequences showing complete identity to the specified sequence.

The term “fiber” as used herein, refers to a fine cord of fibrous material composed of two or more filaments twisted together. By “filament” is meant a slender, elongated, threadlike object or structure of indefinite length, ranging from microscopic length to lengths of a mile or greater. In some embodiments, the fiber is characterized by a size of at least one dimension thereof (e.g., diameter, length). For example, and without limitation, the diameter of the fiber is between 1 μm and 500 μm, between 10 nm and 1 μm, between 20 nm and 100 nm, or between 10 nm and 50 nm. Each possibility represents a separate embodiment of the invention.

As used herein, the terms “fibril” and “fiber” are used herein interchangeably, and refer to any form, intermediate, or final product of a polypeptide chain or a plurality thereof being folded, arranged, and/or organized in a fibril, fiber, or fibrillated form.

In some embodiments, the fiber is characterized by a length ranging from 5 nm to 1.5 μm, 5 nm to 50 μm, 10 nm to 50 μm, 20 nm to 50 μm, 30 nm to 50 μm, 45 nm to 50 μm, 50 nm to 50 μm, 5 nm to 20 μm, 10 nm to 20 μm, 20 nm to 20 μm, 30 nm to 20 μm, 45 nm to 20 μm, 50 nm to 20 μm, 5 nm to 10 μm, 10 nm to 10 μm, 20 nm to 10 μm, 30 nm to 10 μm, 45 nm to 10 μm, 50 nm to 10 μm, 5 nm to 5 μm, 10 nm to 5 μm, 20 nm to 5 μm, 30 nm to 5 μm, 45 nm to 5 μm, 50 nm to 5 μm, 5 nm to 3 μm, 10 nm to 3 μm, 20 nm to 3 μm, 30 nm to 3 μm, 45 nm to 3 μm, 50 nm to 3 μm, 5 nm to 2 μm, 10 nm to 2 μm, 20 nm to 2 μm, 30 nm to 2 μm, 45 nm to 2 μm, or 50 nm to 2 μm, including any range therebetween. Each possibility represents a separate embodiment of the invention.

In some embodiments, the fiber is characterized by a mean diameter ranging from 0.1 nm to 500 nm, 0.5 nm to 500 nm, 1 nm to 500 nm, 2 nm to 500 nm, 4 nm to 500 nm, 5 nm to 500 nm, 0.1 nm to 400 nm, 0.5 nm to 400 nm, 1 nm to 400 nm, 2 nm to 400 nm, 4 nm to 400 nm, 5 nm to 400 nm, 0.1 nm to 400 nm, 0.5 nm to 400 nm, 1 nm to 400 nm, 2 nm to 400 nm, 4 nm to 400 nm, 5 nm to 400 nm, 0.1 nm to 250 nm, 0.5 nm to 250 nm, 1 nm to 250 nm, 2 nm to 250 nm, 4 nm to 250 nm, 5 nm to 250 nm, 0.1 nm to 200 nm, 0.5 nm to 200 nm, 1 nm to 200 nm, 2 nm to 200 nm, 4 nm to 200 nm, or 5 nm to 200 nm, 0.1 nm to 10 μm, including any range therebetween. Each possibility represents a separate embodiment of the invention.

In some embodiments, the fiber is composed of nano-fibrils. In some embodiments, the nano-fibrils have a diameter of e.g., 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 40 nm, about 42 nm, about 44 nm, about 46 nm, about 48 nm, about 50 nm, about 100 nm, about 150 nm, about 180 nm, or about 200 nm, including any value or range therebetween. Each possibility represents a separate embodiment of the invention.

In some embodiments, the disclosed fiber is characterized by a porous structure. In some embodiments, the porous structure is characterized by a porosity of at least 30% (e.g., from 30% to 99%). In some embodiments, the porous structure is characterized by a porosity of at least 50% (e.g., from 50% to 99%). In some embodiments, the porous structure is characterized by a porosity of at least 60% (e.g., from 60% to 99%). In some embodiments, the porous structure is characterized by a porosity of at least 70% (e.g., from 70% to 99%). In some embodiments, the porous structure is characterized by a porosity of at least 80% (e.g., from 80% to 99%). In some embodiments, the porous structure is characterized by a porosity of at least 90% (e.g., from 90% to 99%). In some embodiments, the porous structure is characterized by a porosity of about 90%.

As used herein, the term “porosity” refers to a percentage of the volume of a substance (e.g., a “sponge-like” material) which consists of voids. In another embodiment, porosity is measured according to voids within the surface area divided to the entire surface area (porous and non-porous).

In some embodiments, the polypeptide is characterized by a porosity ranging from 20% to 100%, 15% to 80%, 10% to 75%, 20% to 60%, 25% to 95%, 30% to 70%, 40% to 85%, or 10% to 90%. Each possibility represents a separate embodiment of the invention.

In some embodiments, the polypeptide comprises pores with an average size ranging from 1 μm to 200 μm, 5 μm to 200 μm, 10 μm to 200 μm, 15 μm to 200 μm, 30 μm to 200 μm, 50 μm to 200 μm, 80 μm to 200 μm, 100 μm to 200 μm, 1 μm to 150 μm, 5 μm to 150 μm, 10 μm to 150 μm, 15 μm to 150 μm, 30 μm to 150 μm, 50 μm to 150 μm, 80 μm to 150 μm, 1 μm to 100 μm, 5 μm to 100 μm, 10 μm to 100 μm, 15 μm to 100 μm, 30 μm to 100 μm, or 50 μm to 100 μm, including any range therebetween. Each possibility represents a separate embodiment of the invention.

In some embodiments, the polypeptide is characterized by a tensile strength ranging from 10 MPa to 500 MPa, 15 MPa to 250 MPa, 1 MPa to 200 MPa, 100 MPa to 500 MPa, 40 MPa to 375 MPa, 60 MPa to 1,000 MPa, or 10 MPa to 1,000 MPa. Each possibility represents a separate embodiment of the invention.

In some embodiments, the polypeptide is characterized by a young's modulus ranging from 1 KPa to 15 KPa, 5 KPa to 20 KPa, 0.1 KPa to 5 KPa, 10 KPa to 150 KPa, 100 KPa to 1,500 KPa, 0.01 KPa to 1.5 KPa, 40 KPa to 250 KPa, 25 KPa to 450 KPa, or 1 KPa to 950 KPa. Each possibility represents a separate embodiment of the invention.

In some embodiments, the term “tensile strength” as used herein is the maximum amount of force as measured e.g., in Newton's that a material can bear without or prior to tearing, breaking, necking forming microcracks or fractures.

As used herein the term “Young's modulus” refers to the response of a material to application of tensile stress (e.g., according to any procedure known in the art).

In some embodiments, the polypeptide comprises a cross-linking modification.

As used herein, the term “cross-linking” refers to the process of joining two or more molecules by a covalent bond. In some embodiments, cross-linking is intramolecular (between two groups on a single protein). In some embodiments, cross-linking is intermolecular (between groups on two different proteins). Cross-linking approaches include chemical (glutaraldehyde, isocyanates or carbodiimide based), physical (dehydrothermal, UV irradiation) and enzymatic. In some embodiments, cross linking comprises chemical cross-linking.

In some embodiments, the linker is a rigid linker. In some embodiments, the linker is a flexible linker.

In some embodiments, the polypeptide comprises a chemical cross-linking modification. In some embodiments, cross-linking is performed using an EDC (1-ethyl-3-(3-dimethylaminopropyl)carbodiimide), NHS (N-hydroxysuccinimide) combination.

In some embodiments, crosslinking improves the polypeptide mechanical properties and enhances resistance to degradation.

In some embodiments, the polypeptide is characterized by a crosslinking degree ranging from 1 to 5, 1 to 10, 1 to 15, 1 to 25, 1 to 30, 1 to 50, 1 to 60, 1 to 75, 1 to 90, or 1 to 100. Each possibility represents a separate embodiment of the invention.

In some embodiments, the polypeptide comprises pores with an average size ranging from 50 μm to 500 μm, 80 μm to 500 μm, 90 μm to 500 μm, 100 μm to 500 μm, 150 μm to 500 μm, 200 μm to 500 μm, 50 μm to 250 μm, 80 μm to 250 μm, 90 μm to 250 μm, 100 μm to 250 μm, or 150 μm to 250 μm, including any range therebetween. Each possibility represents a separate embodiment of the invention.

### Polynucleotides

According to some embodiments, the present invention provides a polynucleotide comprising a nucleic acid sequence encoding the polypeptide described herein.

In some embodiments, the polynucleotide comprises a nucleic acid sequence:

(SEQ ID NO: 2)
ATGGGTGGTAGCGGTTATGGCAGCGGTAGCTGGTGCGGCACCACCCCGG
GCAGCTGGTGCGGTACCGGTCCGCCGGGTCCGCCGGGTCCGCCGGGCCC
ACCGGGTCCGCCGGGTCCGCCAGGTCCACCGGGCCCGCCGGGCCCGCCA
GGTCCGCCGGGTCCACCGGGTCCGCCGGGTCGTGATGGTAGCCCGGGTG
GTAGTGGATAGGGAAGTGGA.

In some embodiments, the polynucleotide comprises a nucleic acid sequence:

(SEQ ID NO: 3)
ATGGGTGGTAGCGGTTATGGCAGCGGTAGCTGGTGCGGCACCACCCCGG
GCAGCTGGTGCGGTACCGGTCCGCCGGGTCCGCCGGGTCCGCCGGGCCC
ACCGGGTCCGCCGGGTCCGCCAGGTCCACCGGGCCCGCCGGGCCCGCCA
GGTCCGCCGGGTCCACCGGGTCCGCCGGGCCCACCGGGTCCACCGGGTC
CGCCAGGTCCGCCAGGCCCGCCGGGTCCACCAGGCCCGCCAGGCCCACC
GGGCCCACCAGGCCCGCCAGGTCCACCAGGTCCACCAGGCCCACCAGGT
CCGCCGGGCCCACCGGGTCCGCCAGGCCCGCCAGGTCCGCCAGGCCCAC
CGGGTCCACCGGGCCCGCCAGGCCCGCCGGGTCCGCCAGGCCCACCGGG
CCCGCCGGGTCCACCGGGTCCACCAGGTCCGCCGGGTCCACCAGGTCCG
CCGGGCCCGCCAGGTCCACCGGGCCCACCAGGTCCGCCGGGTCCGCCGG
GTCCGCCGGGTCCGCCGGGTCCGCCGGGTCGTGATGGTAGCCCGGGTGG
TAGTGGATAGGGAAGTGGA.

The terms “polynucleotide” and “nucleic acid” as used interchangeably herein, refer to polymers of nucleotides of any length, and include DNA and RNA, or any combination thereof.

Polynucleotides encoding polypeptides may be obtained from any source including, but not limited to, a cDNA library prepared from tissue believed to possess the polypeptide mRNA and to express it at a detectable level. Accordingly, polynucleotides encoding a polypeptide can be conveniently obtained from a cDNA library prepared from human tissue. The polypeptide-encoding gene may also be obtained from a genomic library or by known synthetic procedures (e.g., automated nucleic acid synthesis).

In some embodiments, the polynucleotide is codon optimized to facilitate or increase translation efficiency in a host cell.

Methods for codon optimization are common and would be apparent to one of ordinary skill in the art, as well as codon preference of various types of cells, e.g., of E. coli.

In some embodiments, the poly nucleotide of the invention comprises a pre-mature stop codon. In some embodiments, the polynucleotide of the invention comprises a codon of an amino acid located in the C terminus of its encoded polypeptide substituted to a stop codon. In some embodiments, the polynucleotide of the invention is modified so as to include a substitution of an amino acid codon to a premature stop codon. In some embodiments, the polynucleotide of the invention is translated to a fully functional polypeptide according to the herein disclosed method. In some embodiments, the polynucleotide of the invention is translated to the polypeptide of the invention when expressed in a host cell in the presence of a non-canonical amino acid, an exogenous amino-acyl tRNA synthase, or any combination thereof. In some embodiments, a polynucleotide of the invention expressed in an endogenous host cell and/or in the absence of any one of a non-canonical amino acid and an exogenous amino-acyl tRNA synthase, does not provide the polypeptide of the invention. In some embodiments, a polynucleotide of the invention expressed in an endogenous host cell and/or in the absence of any one of a non-canonical amino acid and an exogenous amino-acyl tRNA synthase, results in a partial or truncated polypeptide. In some embodiments, the truncated protein is degraded. In some embodiments, the truncated protein is not the polypeptide of the invention.

According to some embodiments, there is provided an artificial vector comprising the polynucleotide of the invention.

In some embodiments, the artificial vector is an expression vector or a plasmid.

In some embodiments, the expression vector further comprises a tRNA/tRNA-synthetase pair comprising a polynucleotide encoding a tRNA and a polynucleotide encoding an amino-acyl tRNA synthetase.

In some embodiments, the tRNA/tRNA-synthetase pair are an orthogonal pair to the cell endogenous tRNAs and aminoacyl-tRNA synthetase.

The term “orthogonal pair” refers to a tRNA/tRNA-synthetase pair which does not occur naturally in the cell. As used herein the terms “tRNA synthase”, “amino-acyl tRNA synthetase” and “aaRS” refer to an enzyme capable of acylating a tRNA with an amino acid or amino acid analog. In some embodiments, the aaRS does not catalyze aminoacylation of an endogenous tRNA. In some embodiments, the aaRS aminoacylate the orthogonal tRNA with a non-canonical amino acid (ncAA).

According to some embodiments the orthogonal tRNA/tRNA-synthetase pair is derived from Eukaryote, Bacteria, or Archaea. According to some embodiments the orthogonal tRNA/tRNA-synthetase pair is derived from an organism selected from: E. coli, Methanocaldococcus jannaschii, Methanosarcina barkeri, Desulfitobacterium hafniense, Methanobacterium thermoautotrophicumLeuRS/Halobacterium sp., Methanosarcina mazei, Saccharomyces cerevisiae, and Bacillus stearothermophilus. According to some embodiments, the orthogonal tRNA/tRNA-synthetase pair is a chimeric pair.

In some embodiments, the amino-acyl tRNA synthetase comprises tyrosyl-tRNA synthase. In some embodiments, the amino-acyl tRNA synthetase is tyrosyl-tRNA synthase. In some embodiments, the tyrosyl-tRNA synthase is a modified or an evolved tyrosyl-tRNA synthase. As used herein, the term “modified” or “evolved” refers to the tyrosyl-tRNA synthase being capable of ligating a ncAA residue to its receptive tRNA. In some embodiments, the ncAA comprises L-DOPA. In some embodiments, the modified or evolved tyrosyl-tRNA synthase is capable of or characterized by being capable of ligating L-DOPA.

The term “expression vector” as used herein, refers to a nucleic acid molecule (e.g., a plasmid, phage, autonomously replicating sequence (ARS), artificial chromosome, yeast artificial chromosome (e.g., YAC)) that can be replicated in a host cell and be utilized to introduce a gene or genes into a host cell. The genes introduced on the expression vector can be endogenous genes (e.g., a gene normally found in the host cell or organism) or heterologous genes (e.g., genes not normally found in the genome or on extra-chromosomal nucleic acids of the host cell or organism). The genes introduced into a cell by an expression vector can be native genes or genes that have been modified or engineered.

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture.

According to some embodiments, the present invention provides a cell comprising: (a) the polypeptide described herein; (b) the polynucleotide described herein; (c) the artificial vector described herein; or (d) any combination of (a) to (c).

In some embodiments, the cell is a prokaryotic cell. In some embodiments, the cell is an Escherichia coli cell. In some embodiments, the E. coli cell comprises any type, strain, or line of E. coli that is sufficient to express the polypeptide of the invention. E. coli types, lines, strains, etc. suitable for heterologous expression are common and would be apparent to one of skill in the art. Non-limiting examples of such cell, include, but are not limited to BL-21, DH5a, or others, such exemplified herein.

According to some embodiments, the present invention provides a composition comprising: (a) the polypeptide described herein; (b) the polynucleotide described herein; (c) the artificial vector described herein; (d) the cell described herein; or (e) any combination of (a) to (d), and an acceptable carrier.

In some embodiments, the composition is in the form of: a sponge, a hydrogel, a hollow fiber tubing, a film, ink, a patch, or a membrane.

In some embodiments, the composition is a porous composition.

As used herein, the term “porous composition” refers to a composition comprising or made of interconnected network of pores. In some embodiments, the composition is a solid composition. In some embodiments, at least a portion of the composition is solid. In some embodiments, the composition comprises a solid portion and a non-solid portion. In some embodiments, a non-solid portion comprises a liquid. In some embodiments, a non-solid portion is a liquid portion.

In some embodiments, the composition being in the form of: a sponge, a hydrogel, a hollow fiber tubing, a film, ink, a patch, or a membrane, further comprises at least one additional polymer. In some embodiments, the at least one additional polymer comprises or is a polysaccharide. In some embodiments, the at least one additional polymer comprises or is alginate.

In some embodiments, a method for producing a composition being in the form of: a sponge, a hydrogel, a hollow fiber tubing, a film, ink, a patch, or a membrane, and at least one additional polymer, comprises mixing the additional polymer and the polypeptide of the invention being in an oxidized state, a fibrillated state, or both. In some embodiments, a method for producing a composition being in the form of: a sponge, a hydrogel, a hollow fiber tubing, a film, ink, a patch, or a membrane, and at least one additional polymer, comprises mixing the additional polymer and the polypeptide of the invention after the polypeptide of the invention has been contacted with oxidizing agent or an oxidizer.

In some embodiments, the method comprises a step before the mixing, comprising contacting the polypeptide of the invention with an effective amount of an oxidizer or an oxidizing agent.

In some embodiments, the composition is for use in any one of: wound healing, three-dimensional (3D) cell culturing, nerve regeneration, tissue regeneration, bone repair, skin graft, cosmetics, 3D printing, cardiac patches, and any combination thereof.

In some embodiments, there is provided a scaffold comprising the composition described hereinabove, wherein the scaffold is in the form of a patch or an implant. In some embodiments, the scaffold is in the form of a sponge.

In some embodiments, the composition, the sponge, the scaffold, or any combination thereof further comprises a polymer. In some embodiments, the polymer is or comprises a saccharide. In some embodiments, a saccharide is or comprises a polysaccharide. In some embodiments, a polymer is or comprises alginate. In some embodiments, the composition, the sponge, the scaffold, or any combination thereof further comprises a mineral. In some embodiments, a mineral is or comprises calcium.

In some embodiments, the scaffold, composition, sponge, or any combination thereof is a porous scaffold, composition, or a sponge.

In some embodiments, the scaffold, composition, sponge, or any combination thereof is characterized by an average pore size ranging between 200 and 800 μm², 200 and 400 μm², 200 and 500 μm², or 200 and 600 μm². Each possibility represents a separate embodiment of the invention. In some embodiments, the scaffold, composition, sponge, or any combination thereof comprises calcium in a concentration ranging between 0.05% and 0.25%, 0.1% and 0.2%, 0.12% and 0.2%, 0.14% and 0.2%, 0.16% and 0.2%, 0.17% and 0.2%, or 0.12% and 0.16%. Each possibility represents a separate embodiment of the invention.

In some embodiments, the scaffold, composition, sponge, or any combination thereof is characterized by increased cell viability thereon increased cell adhesion thereto, or both. In some embodiments, increased is compared to a control scaffold, composition, sponge, or any combination thereof.

In some embodiments, the control does not comprise or is devoid of a polypeptide comprising the amino acid sequence set forth in SEQ ID NO: 1 or SEQ ID NO: 4. In some embodiments, the control comprises a polypeptide devoid of L-DOPA.

In some embodiments, increased comprises at least 5%, 20%, 50%, 100%, 250%, 500%, or 1,000% increase (such as compared to control), or any value and range therebetween. Each possibility represents a separate embodiment of the invention. In some embodiments, increased comprises 1-50%, 5-50%, 20-60%, 10-100%, 5-200%, 20-100%, 50-400%, 100-500%, 250-600%, 500-900%, or 100-1,000% increase (such as compared to control). Each possibility represents a separate embodiment of the invention.

In some embodiments, there is provided a coating comprising the composition described herein. In some embodiments, the composition is in the form of a coating on prosthetics, films, nonwovens, or meshes.

### The Method

According to some embodiments, the present invention provides a method for producing the polypeptide described hereinabove. In some embodiments, the method comprises the steps of (a) providing a transformed bacterial cell comprising the artificial vector described hereinabove; and (b) culturing the transformed cell.

In some embodiments, the method comprises contacting a polypeptide comprising at least 80% homology to the amino acid sequence: MGSGYGSGSWCGTTPGSWCGT-[GPP]ₙ-GRDGSPGGSGYGSG with an effective amount of a tyrosinase enzyme, or a functional analog thereof. In some embodiments, any one of X₁ and X₂ is Tyrosine or L-DOPA. In some embodiments, when X₁ is Tyrosine X₂ is L-DOPA, and/or vice versa. In some embodiments, both X₁ and X₂ are L-DOPA.

In some embodiments, a functional analog refers to any enzyme, polypeptide, protein, agent, or the like, which is not tyrosinase, as long as it has an activity being capable of modifying, catalyzing, converting, or any combination thereof, tyrosine to L-DOPA. In some embodiments, a tyrosinase comprises tyrosine hydroxylase. In some embodiments, tyrosine hydroxylase is a functional analog of tyrosinase.

Types and sources of tyrosinase enzymes, and the like, are common and would be apparent to one of ordinary skill in the art.

In some embodiments, the culturing comprises contacting the transformed bacterial cell with a sufficient amount of L-DOPA, thereby producing the polypeptide described hereinabove.

In some embodiments, the method further comprises a step comprising transforming or transfecting the artificial vector into a bacterial cell, thereby providing the transformed bacterial cell. In some embodiments, the transforming or transfecting step preceded step (a) as described herein.

In some embodiments, the method further comprises a step comprising isolating, purifying, or both, the produced polypeptide. In some embodiments, the step comprising isolating, purifying, or both, the produced polypeptide proceeds step (b) as described herein.

In some embodiments, the transformed cell further comprises a transfer RNA (tRNA) synthase capable of ligating L-DOPA to a tRNA molecule.

In some embodiments, the tRNA synthase is a modified tyrosyl-tRNA synthase.

In some embodiments, the tRNA substrate of the modified tyrosyl-tRNA synthase comprises an anti-codon comprises a nucleic acid sequence of CUA. In some embodiments, the modified tyrosyl-tRNA synthase ligates L-DOPA to the tRNA substrate comprising an anti-codon comprising a nucleic acid sequence of CUA.

In some embodiments, the method further comprises a step preceding step (a) comprising transforming or transfecting the bacterial cell with a polynucleotide comprising any one of: a nucleic acid sequence of the tRNA molecule, a nucleic acid sequence encoding the tRNA synthase, and both.

In some embodiments, the polynucleotide comprises a plurality of polynucleotide types. In some embodiments, a first type of the plurality of polynucleotide types comprises a nucleic acid sequence of the tRNA molecule, and wherein a second type of the plurality of polynucleotide types encodes the tRNA synthase.

According to some embodiments, the present invention provides a composition comprising a plurality of molecules of the polypeptide produced according to the method described herein, and an acceptable carrier.

In some embodiments, at least 80%, at least 85%, at least 89%, at least 90%, at least 92%, at least 95%, at least 98%, or at least 99% of the plurality of molecules of the polypeptide comprise a L-DOPA, or any value and range therebetween. Each possibility represents a separate embodiment of the invention.

In some embodiments, 100% of the plurality of molecules of the polypeptide comprise a L-DOPA. In some embodiments, the plurality of molecules of the polypeptide comprising a L-DOPA is devoid of a polypeptide devoid of L-DOPA.

In some embodiments, the cell is suspended in a medium. In some embodiments, the cell is grown in the medium. In some embodiments, the medium is a cell culture medium suitable for growth and maintenance of the cell. In one embodiment, the cell culture medium is optimized for cell growth.

As used herein, “cell culture medium” refers to any medium, liquid, semi solid, or solid, which enables cells proliferation. Cell culture media are known in the art and can be selected, depending on the type of cell to be grown. For example, a cell culture medium for use in growing cells is Luria-Bertani broth (LB; Miller's broth). In some embodiments, the cell is cultured under effective conditions, which allow for increased yield of production, e.g., of the polypeptide of the invention, from the culture. Non-limiting examples for increased yield include, but are not limited to, increased gene expression, protein production and/or secretion, molecule biosynthesis, proliferation, others, or any combination thereof. In some embodiments, effective culture conditions include, but are not limited to, effective media, bioreactor, temperature, pH and oxygen conditions that permit for increased production yield. In one embodiment, an effective medium refers to any medium in which a cell is cultured to produce a polypeptide of the invention. In some embodiments, a cell culture medium typically includes an aqueous solution having assimilable carbon, nitrogen and phosphate sources, and appropriate salts, minerals, metals and other nutrients, such as vitamins. In some embodiments, a cell can be cultured in conventional fermentation bioreactors, shake flasks, test tubes, microtiter dishes and petri plates. In some embodiments, culturing is carried out at a temperature, pH and oxygen content appropriate for bacteria, such as E. coli. In some embodiments, culturing conditions are within the expertise of one of ordinary skill in the art.

As used herein, the term “carrier”, “excipient”, or “adjuvant” refers to any component of a composition or pharmaceutical composition that is not the active agent. As used herein, the terms “acceptable carrier” and “pharmaceutically acceptable carrier” refer to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Various embodiments and aspects of the present invention as delineated hereinabove and as claimed in the claims section below find experimental support in the following examples. Reference is now made to the following examples, which together with the above descriptions illustrate some embodiments of the invention in a non-limiting fashion.

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Example 1

### Incorporation of L-DOPA in Multiple Sites of Collagen Polypeptides

The following genetic constructs for the expression of collagen polypeptide in E. coli were designed and constructed: (1) pET15b His-MBP-GPP12 (pGPP12); (2) pET15b His-MBP-GPP50 (pGPP50); and (3) pBEST His-MBP-GPP12 (pBEST GPP12).

In both expression systems (pET15b and pBEST) collagen polypeptide (12 or 50 repetitions of [GPP] sequence) are fused to a 6×His tag and a maltose-binding protein (MBP) tag at the N-terminus. Both tags assist in the identification and purification stages and can be removed by the Tobacco Etch Virus (TEV) protease during collagen polypeptide purification. In the pBEST construct, protein expression is regulated by a constitutive strong promoter, this system is optimized for unnatural amino acid (UAA) incorporation. In the pET15b constructs, protein expression is regulated by the inducible lac operon.

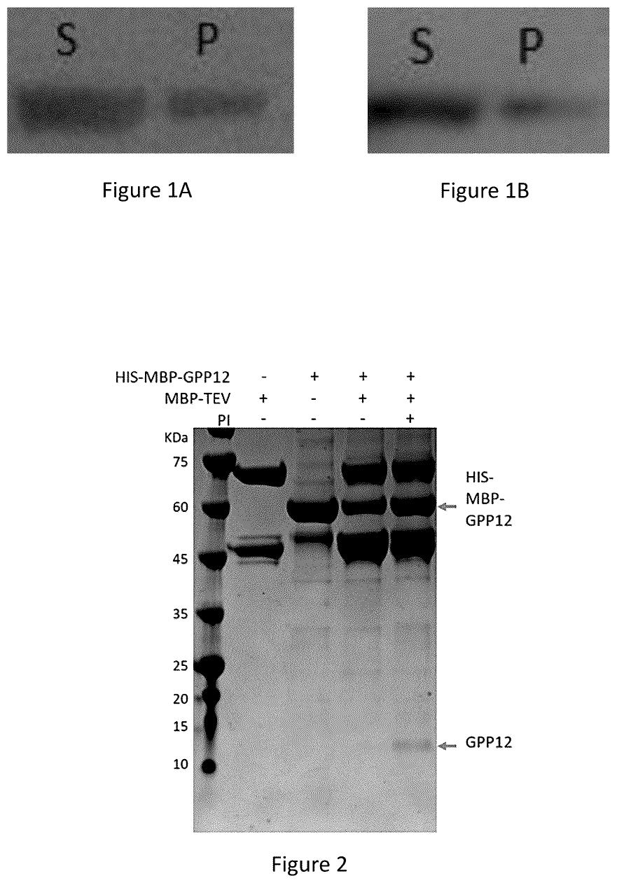

The genetic constructs pGPP12 and pGPP50 were tested for protein expression in BL21 E. coli strain. Full complex expression (His-MBP-[GPP]ₙ, n=12 or 50) was verified through anti-His western blot analysis (FIGS. 1A-1B), intense bands were visible in the supernatant phase after cell lysis, which corresponded with the correct size of the peptides.

After His-MBP-[GPP]ₙ expression was validated in BL21 cells, a large-scale purification procedure, which included the following steps was conducted-BL21 cells were harvested from the growth medium and lysed through sonication. After centrifugation, the supernatant was loaded onto an amylose resin and the eluted sample was further loaded onto Ni-NTA for further purification.

TEV protease was used to cleave fusion tags (FIG. 2). Cleavage efficiency was low and attempts to recover GPP12 from the proteins solution through chromatography resulted in degradation of GPP12 in the process.

For the UAA incorporation, TAG (a stop codon) mutation was inserted successfully by mutagenesis instead of the 5th codon in the collagen peptide, thus coding for the replacement of tyrosine (Y5) (in all constructs).

Next, an orthogonal translation system (OTS) was inserted into BL21 and C321 cells along with the expression constructs pGPP12 and pGPP50 or pBEST GPP12, respectively (His-MBP-[GPP]ₙ Y5 mutants), cells were grown in the presence of 0-4 mM L-DOPA. L-DOPA incorporation resulted in a truncated form in all expression systems.

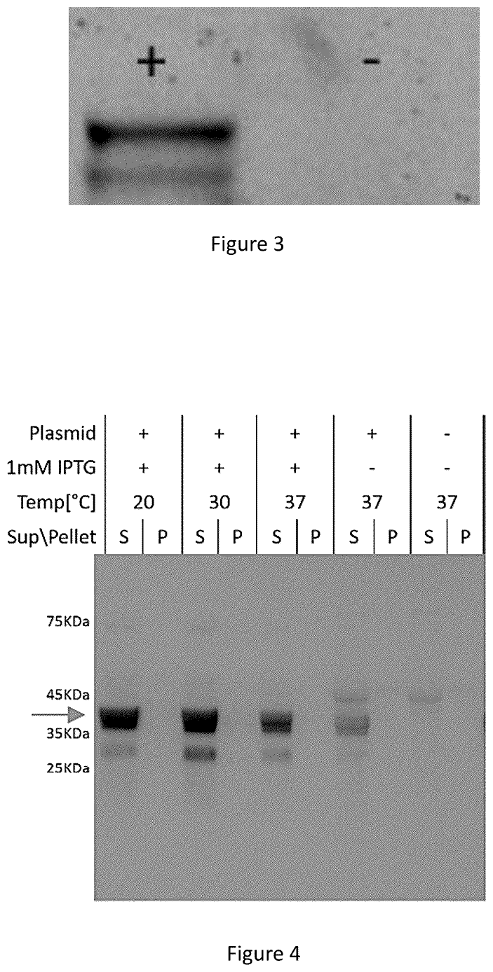

Propargyl-L-lysine (PrK) was incorporated to validate that Y5 is a compatible site for UAA incorporation. The incorporation was confirmed through a click reaction to TAMRA-Az linker (FIG. 3).

With the expression system used, the expression and purification of L-DOPA collagen polypeptides failed. To resolve this issue, new genetic constructs for the expression of collagen polypeptides in E. coli were designed and constructed: (a) pET15b GPP12-Mxe-CBD; and (b) pBEST GPP12-Mxe-CBD.

In these constructs collagen polypeptides (GPP12) were expressed while fused at their C-termini to Mxe-CBD. Mxe, is an Intein, a sequence derived from Mycobacterium xenopi that can be cleaved under reducing conditions, and CBD is a chitin-binding domain that serves as a tag and can also bind to a chitin resin. In this system, can be utilized for the purification of complex GPP12-Mxe-CBD on a chitin resin and perform an on-column cleavage, resulting in a purified collagen polypeptide in the elution fraction.

Collagen polypeptide expression in E. coli (BL21 pET15b GPP12-Mxe-CBD) was verified through anti-CBD western blot (FIG. 4). After induction with IPTG, the bacteria were incubated at different temperatures. At 20° C., it was highly evident that the full complex GPP12-Mxe-CBD was highly expressed as identified in the supernatant phase (FIG. 4, indicated by an arrow).

After GPP12-Mxe-CBD expression was validated in BL21, a large-scale purification procedure, which included the following steps was conducted-BL21 cells were harvest from the growth medium and lysed through sonication or French press. Alternatively, cells were extracted using Bugbuster. After centrifugation, the supernatant was loaded to chitin resin. On-column cleavage was conducted and the eluted sample, GPP12 without Mxe-CBD, was collected.

GPP12 purification was validated using matrix-assisted laser desorption ionization-time of flight (MALDI-TOF) mass spectrometry (FIG. 5), the highest observed peak was at 6,118.580 Da while GPP12 was calculated to be 6,115.59 Da. The ca. 3 Da discrepancy could be attributed to the error of measurement using MALDI-TOF mass-spectrometry.

For the UAA incorporation, TAG (a stop codon) mutation was inserted successfully by mutagenesis to result in the following mutants of the collagen polypeptide GPP12: (i) Y5; (ii) Y68; and (iii) Y5 and Y68.

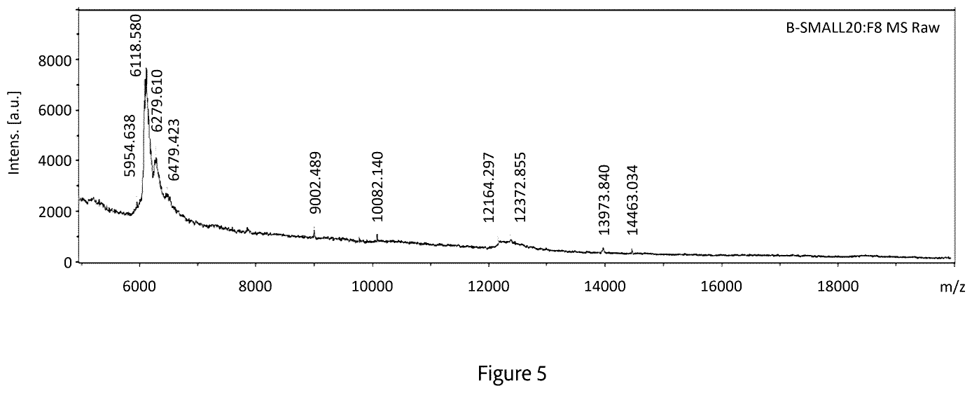

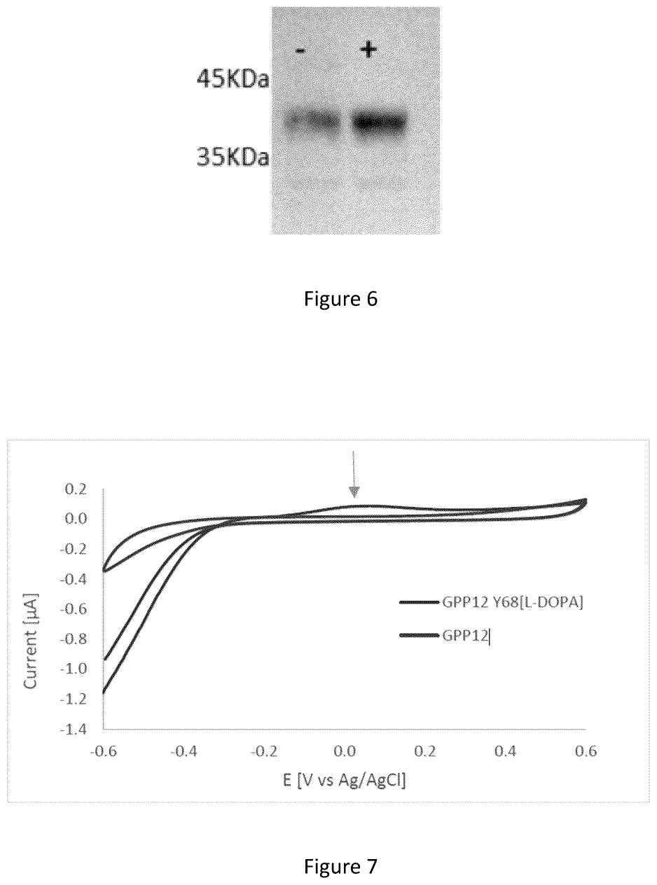

OTS was transformed into BL21 cells along with the mutated expression constructs (pET15b GPP12-Mxe-CBD Y68). L-DOPA incorporation at site Y68 on collagen polypeptide was validated with anti-CBD western analysis (FIG. 6), an intense band was visible when cells were grown in the presence of 2 mM L-DOPA. Nonetheless, a faint band was also observed in the absence of L-DOPA (suggesting that tyrosine or other natural amino acid may still be incorporated).

GPP12 Y68[L-DOPA] was purified according to same method described for GPP12. Cyclic voltammetry was used to detect L-DOPA presence in GPP12 Y68[L-DOPA] (FIG. 7). Both GPP12 and GPP12 Y68[L-DOPA] were dried on a bare glassy carbon electrode (GCE) under argon flow before measurements. All measurements were conducted in PBS pH 7.4, under argon, scan rate 0.01 V/see, using a graphite rod as a counter electrode, Ag/AgCl as a reference electrode. In comparison to GPP12, an oxidation peak (FIG. 7, indicated by an arrow) is observed at E=0.06 V for GPP12 Y68[L-DOPA] an indication of the incorporation of L-DOPA into the peptide.

### Example 2

### Validation of L-DOPA Incorporation

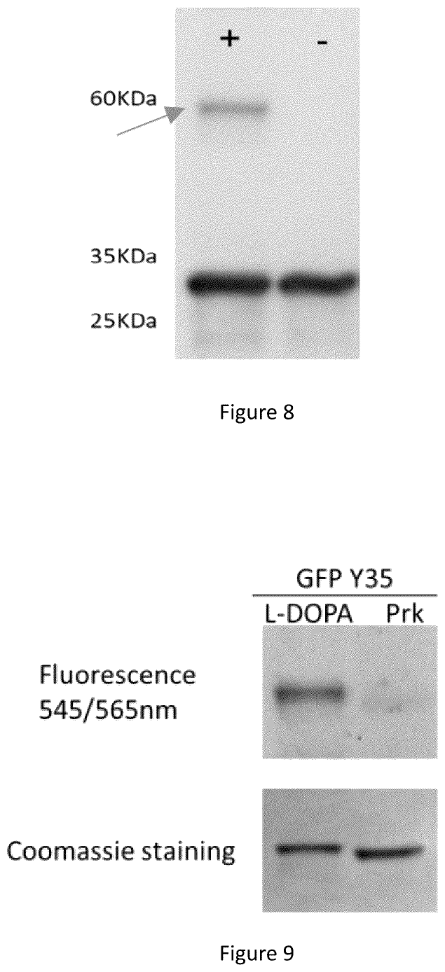

To verify L-DOPA incorporation into proteins according to the herein described method, the same OTS was used to successfully incorporate L-DOPA to a tyrosine located at position 35 (Y35) of the green fluorescence protein (GFP; FIG. 8). GFP dimers (FIG. 8, indicated by an arrow) were observed only when the bacteria with the OTS was grown in the presence of 2 mM L-DOPA. This unique feature is thought to be attributed to the L-DOPA quinone interactions, driving a cross-linking between two neighboring proteins containing L-DOPA, upon oxidation.

GFP Y35 [L-DOPA] was purified on a Ni-NTA chromatography. The purified protein was oxidized with sodium periodate and mixed with a TAMRA-amine fluorophore. The conjugation between the oxidized catechol group of L-DOPA and the amine group in the fluorophore was analyzed by sodium dodecyl sulfate poly acryl amide gel electrophoresis (SDS-PAGE) and fluorescence imaging at 545/565 nm (FIG. 9). The same reaction between TAMRA-amine and purified GFP Y35 [PrK] showed no fluorescence, thus, confirming the specificity of the conjugation with the genetically incorporated L-DOPA. Coomassie staining confirmed the presence of GFP in both samples.

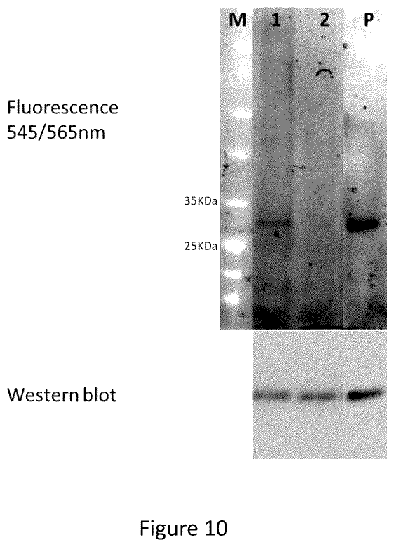

To ensure the specificity of the conjugation, TAMRA-amine was mixed with the oxidized supernatant phase of bacterial lysate. DH5a cells containing pBEST Pr1 deGFP Y35 and pAC-DOPA plasmids, for the expression of GFP Y35 [L-DOPA] were cultured and incubated over night with or without 2 mM of L-DOPA. A clear and a single band corresponding to the molecular weight of GFP was evident in the fluorescent gel for the bacterial sample that was grown in the presence of 2 mM L-DOPA (FIG. 10). Anti-His western blot confirmed the presence of GFP in all samples.

### Example 3

### Secondary Structure Characterization of Collagen L-DOPA Polypeptides

The purified collagen-mimicking peptides underwent mass analysis using liquid chromatography mass spectrometry (LCMS), providing precise mass for each variant, and confirming the fidelity of peptide expression and purification according to design. The efficiency of L-DOPA incorporation was determined to be a minimum of 31% (Table 1).

TABLE 1
Mass spectra of GPP12 & GPP12Y68[L-DOPA].
Expected and deconvoluted mass of each peptide.

| | Expected | Experimental | | |
|---|---|---|---|---|
| | molecular | molecular | Fractional | |
| Peptide | weight [Da] | weight [Da] | abundance | Fit |
| GPP12 | 6246.79 | 6110.73 | 62.68% | GPP without 1^{st} |
| | | | | Met |
| | | 6244.71 | 37.32% | GPP12 |
| GPP12Y68[L- | 6262.79 | 6243.70 | 68.81% | GPP12 |
| DOPA] | | 6260.73 | 31.19% | GPP12Y68[L- |
| | | | | DOPA] |

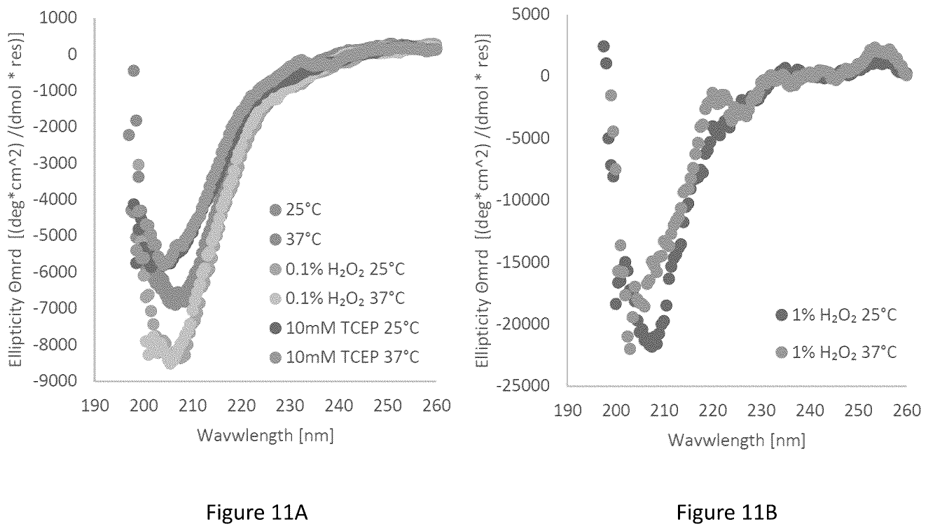

GPP12 secondary structure was examined using circular dichroism (CD) spectroscopy (FIGS. 11A-11B). In CD spectra, a triple helix is determined when the ellipticity has a maximum of ~5,000 [(deg×cm²)/(dmol×res)] at 220-230 nm, and a minimum of ~−5,000 to −20,000 [(deg×cm²)/(dmol×res)] at 190-210 nm.

From FIGS. 11A-11B it can be shown that GPP12 exhibited a triple helix secondary structure. The polypeptide contains two cysteine residues, which under oxidation conditions form disulfide bonds that act as fibrillation cores. In the presence of H₂O₂, (to generate oxidative conditions) the secondary structure of GPP12 was more compatible with a triple helix rather than in the presence of TCEP (tris(2-carboxyethyl) phosphine), which induces reducing conditions. This phenomenon increased with the rise in H₂O₂% (FIG. 11B). The polypeptides' ellipticity did not change in response to temperature shift between 25° C. to 37° C. during the measurements, which implies the structural stability in these temperatures.

### Example 4

### Ability of Collagen L-DOPA Polypeptides Variants to Self-Assemble

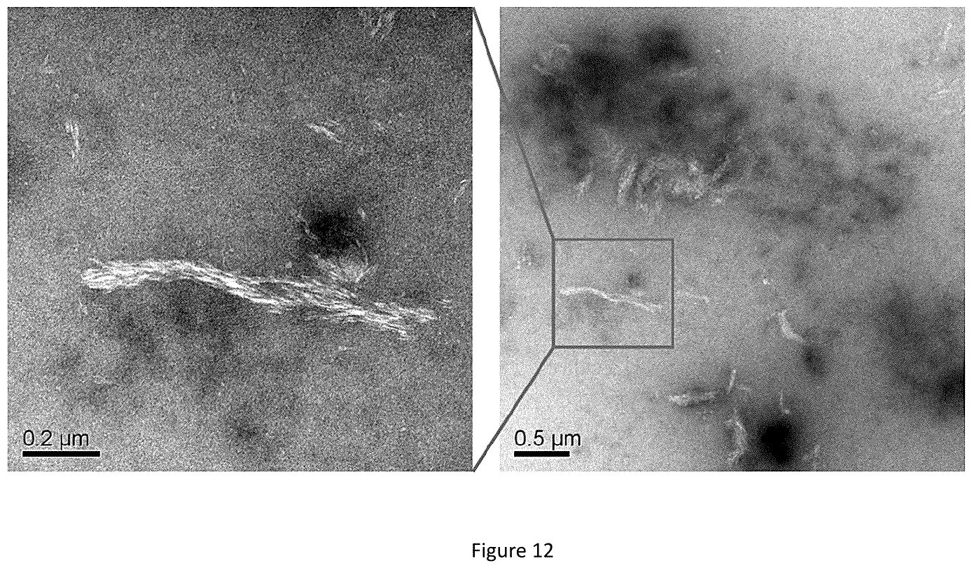

GPP12 fibrils were visualized using a transmitted electron microscope (TEM) (FIG. 12). At a concentration of 0.2 mg/mL, GPP12 peptides began to self-assemble to fibrils. The longest fibril observed was ~0.92 μm long, while the dimension of a natural single tropocollagen helix is 1.5 nm.

Further, the inventors examined GPP12 fibrillation dynamics at a greater concentration. Specifically, 4 experimental groups of 3.8 mg/mL (PBS, pH 7.4) of GPP12 were examined and observed using TEM.

The samples were as follows: (1) Fresh-without any treatment; (2) 7 days incubation at 37° C.; (3) Over-night incubation at 37° C. with 0.1% H₂O₂; and (4) 7 days incubation at 37° C. with 0.1% H₂O₂.

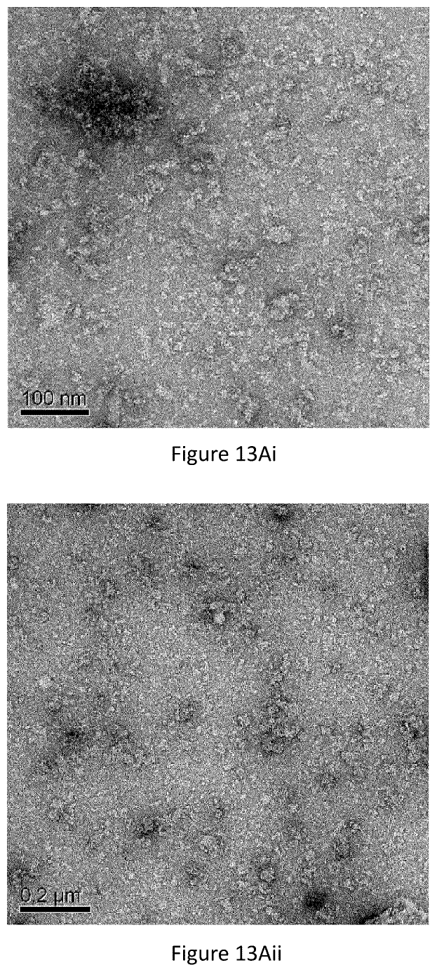

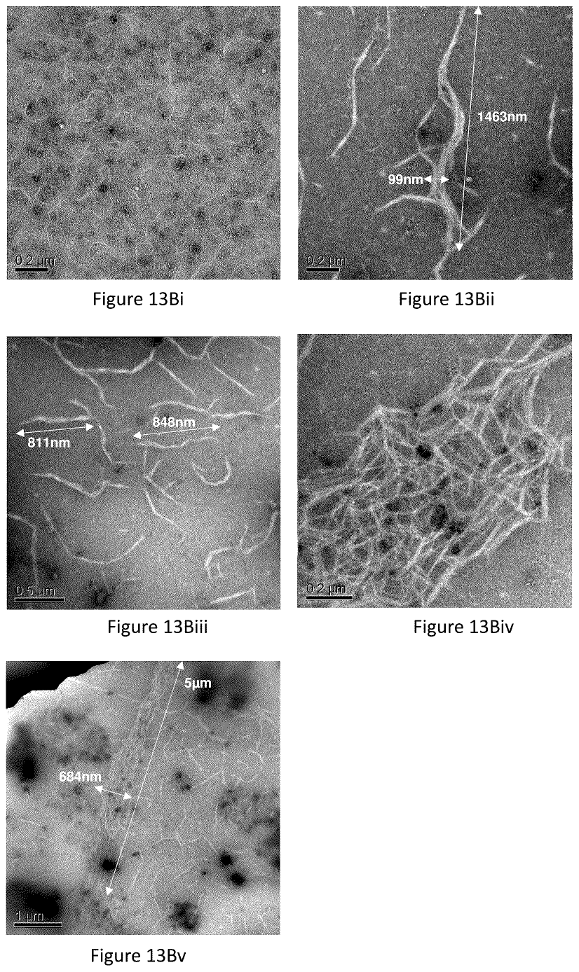

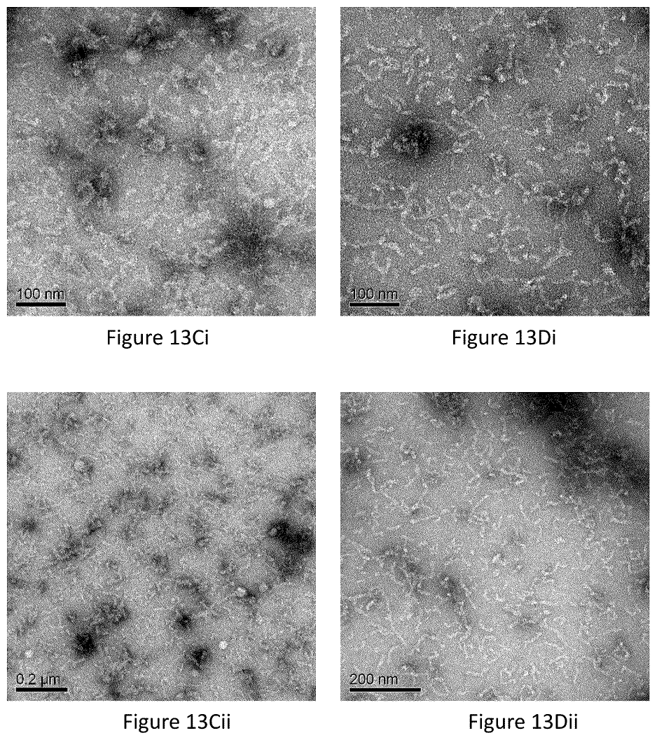

In the control group (sample 1), the inventors observed unorganized material (FIGS. 13Ai-13Aii).

In experimental group2, fibrils and fibers were assembled. FIGS. 13Bi-13Bv, present different regions of the TEM grid (same analysis). In this analysis both small and large fibrils were identified (FIGS. 13Bi-13Biii), as well as a fibrous network (FIGS. 13Biv-13Bv).

After treating the GPP12 peptide with 0.1% H₂O₂, only small fibrils were detected, regardless of the incubation time (FIGS. 13C-13D).

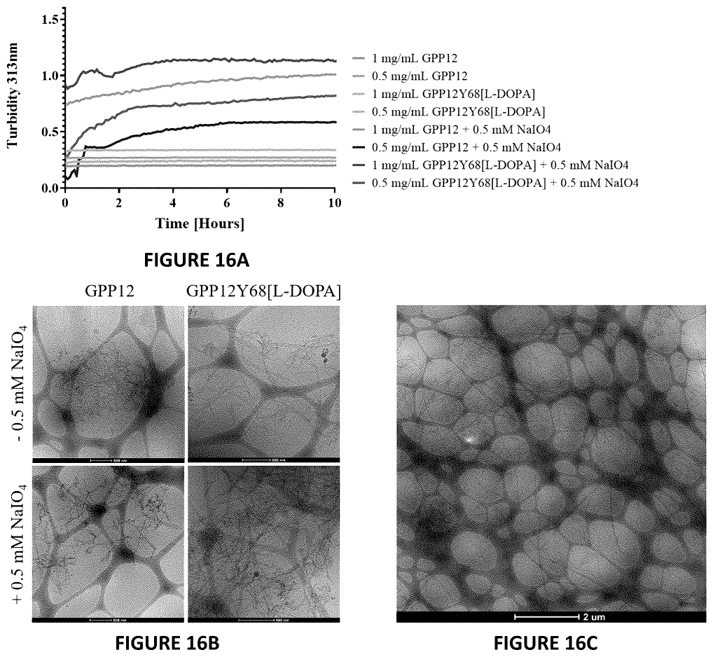

The current peptides are engineered to mimic collagen, a protein that naturally self-assembles into fibrils and subsequently forms robust fibers network that play a pivotal role in supporting cell adhesion and proliferation within the extracellular matrix (ECM). Within the current peptide design, the inventors have incorporated a specific site responsible for fibrillation, and the regulation of this process is achieved through the oxidation of cysteine residues. By monitoring changes in turbidity, the inventors were able to observe how oxidation can effectively control the self-assembly of our peptides (FIG. 16A). When the peptides were exposed to NaIO₄, the inventors observed an increase in turbidity, indicating the initiation of fibrillation. The presence of L-DOPA enhanced this process, as L-DOPA has the capacity to undergo cross-linking under oxidative conditions, further promoting fibrillation. Following an overnight incubation with NaIO₄, GPP12Y68[L-DOPA] exhibited the formation of longer and wider fibrils in comparison to GPP12 both with and without the presence of the oxidizer, as well as GPP12Y68[L-DOPA] without the oxidizer (FIG. 16B). The concentration of the peptide also plays a significant role in the process. Lower peptide concentrations resulted in fewer nucleation sites, ultimately leading to the formation of longer fibrils (FIG. 16C).

### Example 5

### Characterization of Collagen Scaffold Properties as a Biomaterial

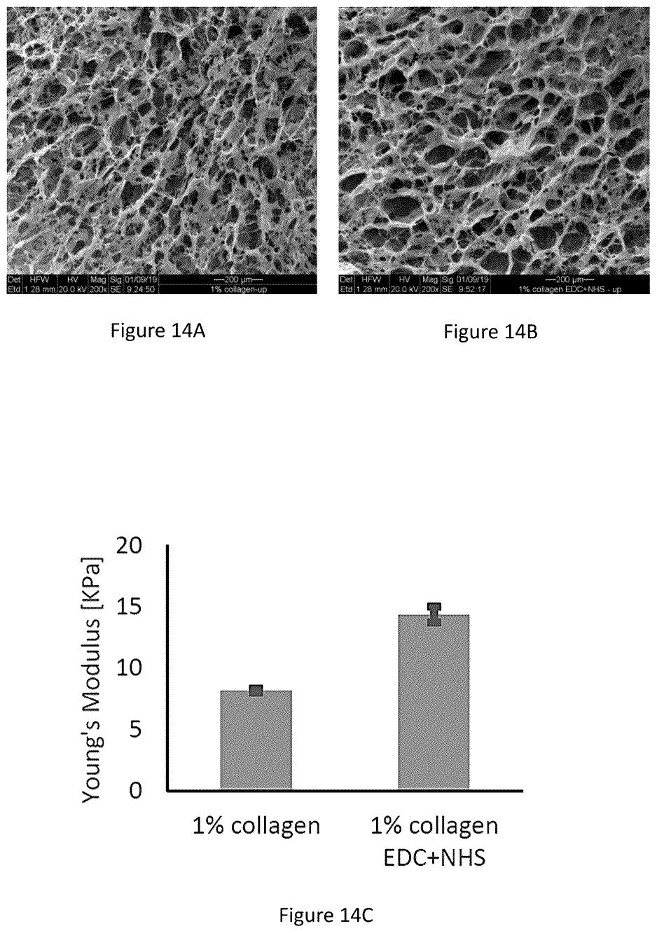

For future experiments with L-DOPA enriched engineered collagen scaffolds, collagen extracted from bovine Achilles tendon was selected to serve as a control. 1% collagen sponge scaffolds were prepared according to standard procedure using ethyl(dimethylaminopropyl) carbodiimide-N-Hydroxysuccinimide (EDC-NHS) chemical cross-linking. Porous structure and mechanical strength were tested (FIGS. 14A-14C). Chemical cross-linking with EDC-NHS resulted in a more organized porous structure (FIG. 14B), the pores were larger and more defined rather when cross-linking did not take place (FIG. 14A). Young's modulus measurement showed that after cross-linking the scaffolds were also stronger (FIG. 14C).

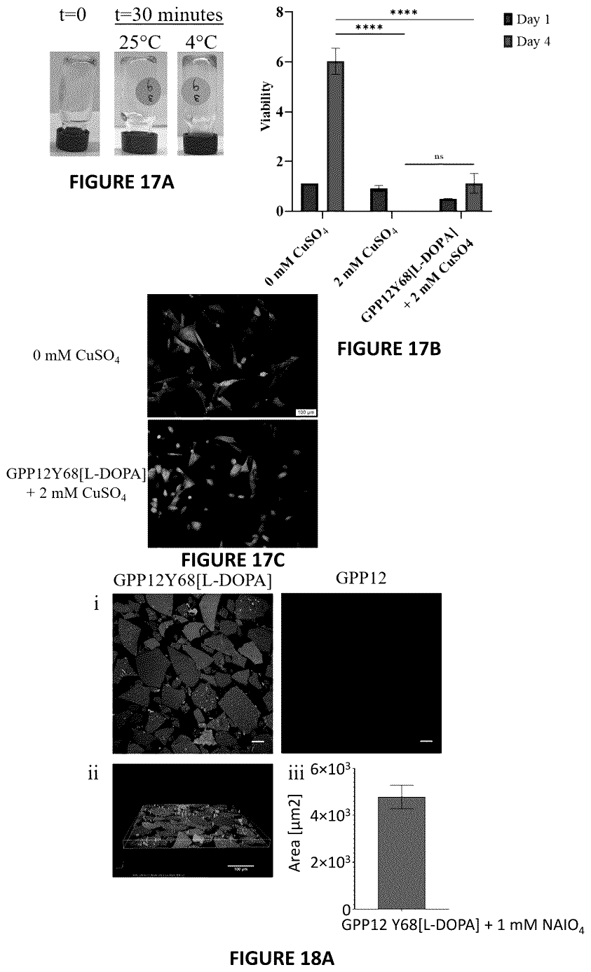

In the current study, the inventors aimed to employ collagen-mimicking peptides as a biomaterial. The current objective was to generate hydrogel solely using the peptides of the invention. To achieve this, the inventors explored the requisite conditions under which the peptides of the invention could self-assemble into a hydrogel matrix. The inventors identified a condition wherein the current peptides exhibit self-assembly into a hydrogel. Unfortunately, this hydrogel proved to be unstable under 37° C., the temperature conditions necessary for cell growth. Furthermore, the formation of the hydrogel necessitates a high concentration of Cu ions, which were toxic for the cells. (FIG. 17).

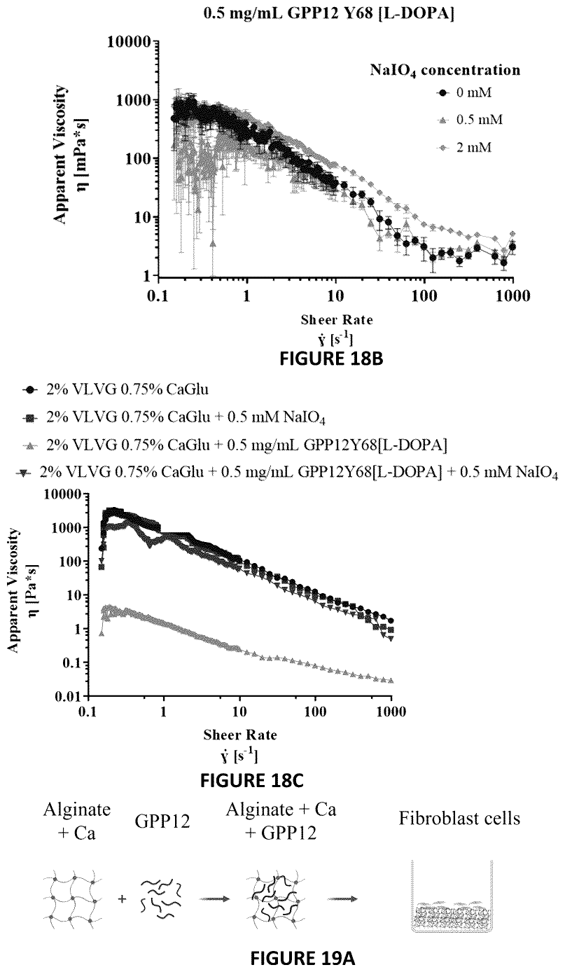

Therefore, the inventors pursued an alternative approach for the hydrogel preparation from the peptides. L-DOPA has the capacity to undergo cross-linking under oxidative conditions. The inventors discovered that, in the presence of NaIO₄, GPP12Y68[L-DOPA] could assemble into thin sheets. Visualization of these sheets was achieved through TAMRA amine staining under a confocal microscope (FIG. 18A). It is important to note that TAMRA amine staining selectively identified L-DOPA-containing peptide GPP12Y68[L-DOPA], distinguishing it from GPP12. Using this oxidizer, the inventors could only produce thin films instead of 3D hydrogel or scaffolds, attributed to the low viscosity of the peptide (FIG. 18B). As part of the inventors efforts to establish a 3D cell culture using the peptide, the inventors tested the viscosity of a cross-linked alginate solution mixed with GPP12Y68[L-DOPA] and NaIO₄. Interestingly, the inventors observed that in the absence of oxidizer, the peptide of the invention reduced the viscosity of the alginate solution. This effect likely arises from L-DOPA's ability to bind to calcium ions, thereby interfering with the cross-linking of alginate (FIG. 18C). Upon oxidation, GPP12Y68[L-DOPA] undergoes a process of cross-linking where L-DOPA residues form bonds that do not interfere with the cross-linking of alginate. Upon identifying the optimal combination for the formation of a stable 3D hydrogel scaffold using the current L-DOPA collagen-mimicking peptides, the inventors proceed to finalize this characterization step.

### Example 6

### Comparison of L-DOPA Incorporation into Different Protein Residues

Thin layer film was prepared in a 96 well plate by mixing 0.6% LVG (alginate) and different peptide variants. The film was dried for 1 hour in a biological hood (30 min exposure to UV). Human fibroblast cells expressing GFP (HFGFP) were cultured on the thin films.

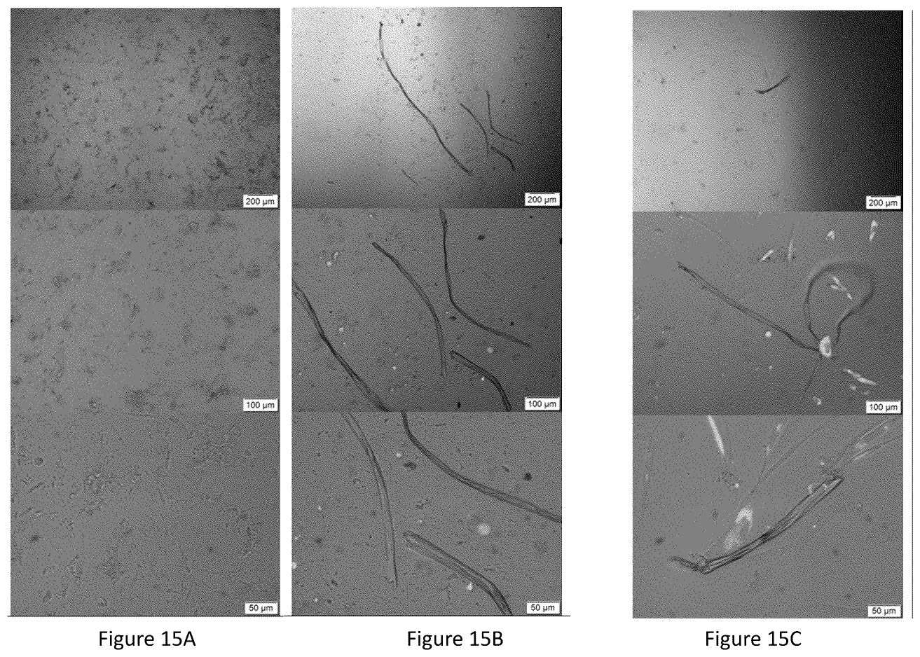

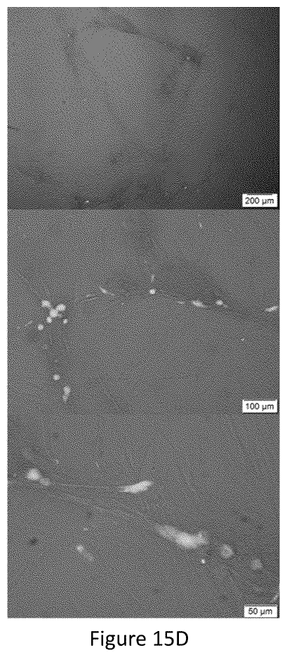

As a control, 0.6% LVG+collagen Type I (extracted rat tail) was used and the behaviour of the cells on the different films was examined. The cells were counted and distributed equally to seed 5,000 cells per well. Bright-field and fluorescent images were taken after 3 days incubation (FIGS. 15A-15D).

After L-DOPA incorporation, both GPP12 Y5 [L-DOPA] and GPP12 Y68[L-DOPA] exhibited long and twisted fibres (up to 1.5 mm) while for GPP12 only fibrils (up to 100 μm) were observed.

It was observed that, fibrilization in suspension occurred immediately once L-DOPA was incorporated into the peptide, while in the absence of L-DOPA the process was slower (up to 7 days).

An advantage of L-DOPA incorporation in C-terminus Y68 was observed in relation to N-terminus Y5. Fibroblasts cells remained round in the presence of GPP12 and GPP12 Y5 [L-DOPA] (FIG. 15B). In sharp contrast, the cells stretched in the presence of GPP12 Y68[L-DOPA] (FIG. 15C) as in the positive control with collagen type I (FIG. 15D), thus showing that not any tyrosine substitution is suitable for providing sufficient fibrillation and/or adhesion, but rather C terminal modification, as shown by Y68[L-DOPA] is required to provide more appropriate properties for the cells to adhere to the fibre (FIG. 15C).

### Example 7

### Validation and Characterization of Cell Adhesion Properties of the L-DOPA Collagen Scaffold

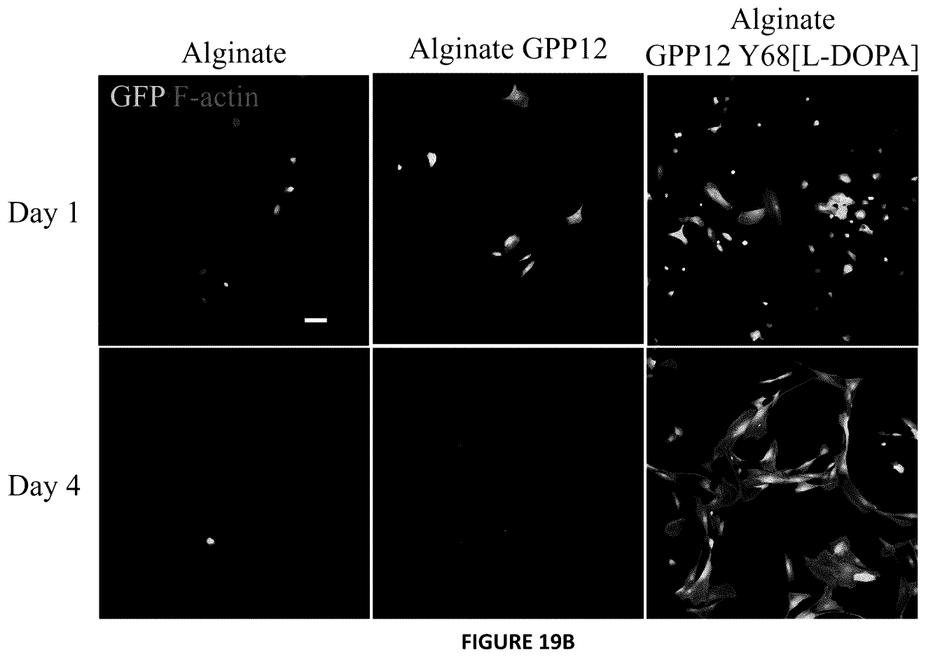

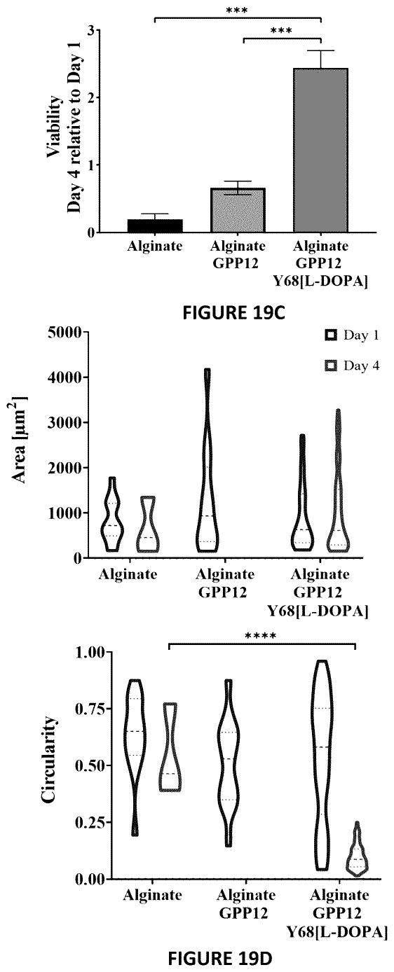

In FIG. 19 the inventors demonstrated that human fibroblasts cells expressing GFP (HFGFP) exhibited growth on a thin film/hydrogel composed of the peptides of the invention and alginate. Furthermore, cell proliferation and spreading were observed upon the incorporation of L-DOPA into the peptide.

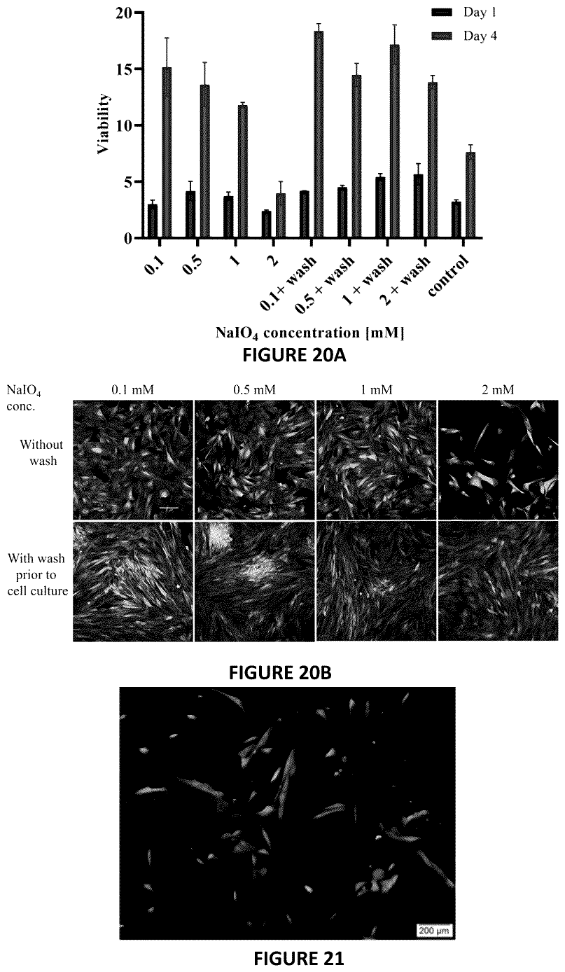

In Example 5, it was reported that 2D hydrogel sheets were successfully generated from oxidized GPP12Y68[L-DOPA]. Considering the potential cytotoxic effects of the oxidizer, the inventors verified whether HFGFP cells could successfully proliferate and spread on these sheets (FIG. 20). In summary, HFGFP cells demonstrated adhesion, proliferation, and spreading on 2D films incorporating the L-DOPA collagen-mimicking peptides of the invention. Upon successful construction of a 3D scaffold using the L-DOPA collagen-mimicking peptides of the invention, the inventors further investigate the behavior of other mammalian cells, assessing parameters such as adhesion, proliferation, and spreading.

### Example 8

### Fabrication of Alginate-L-DOPA Collagen Scaffolds, and Characterization of Cell Adhesion Properties

To fabricate a three-dimensional sponge scaffold utilizing the current collagen-mimicking peptides, the inventors employed EDC coupling to conjugate the N-terminus of fibrillized collagen-mimicking peptides with the carboxylic groups within alginate (LVG).

To validate the effectiveness of the coupling process, the inventors conducted X-ray photoelectron spectroscopy (XPS) analysis on the resulting product. LVG exhibited no detectable nitrogen (N) atoms before the reaction. However, following the reaction with fibrillized GPP12Y68[L-DOPA] or GPP12, the inventors observed an increase in nitrogen atom content of 4.01% or 4.34%, respectively (Table 2).

TABLE 2
XPS analysis of LVG, LVG-GPP12Y68[L-
DOPA], and LVG-GPP12.

| | LVG | LVG-GPP12Y68[L-DOPA] | LVG-GPP12 |
|---|---|---|---|
| C1s | 52.52% | 51.99% | 53.71% |
| N1s | — | 4.09% | 4.34% |
| Na1s | 6.86% | 3.51% | 4.02% |
| O1s | 40.61% | 40.04% | 37.22% |

HFGFP cells were cultured on thin films composed of 1% LVG-GPP12Y68[L-DOPA] cross-linked with 0.14% calcium. Following a 7-day incubation period, observable cell spreading was evident (FIG. 21), serving as an indicator for the presence and effectiveness of GPP12Y68[L-DOPA] in the film.

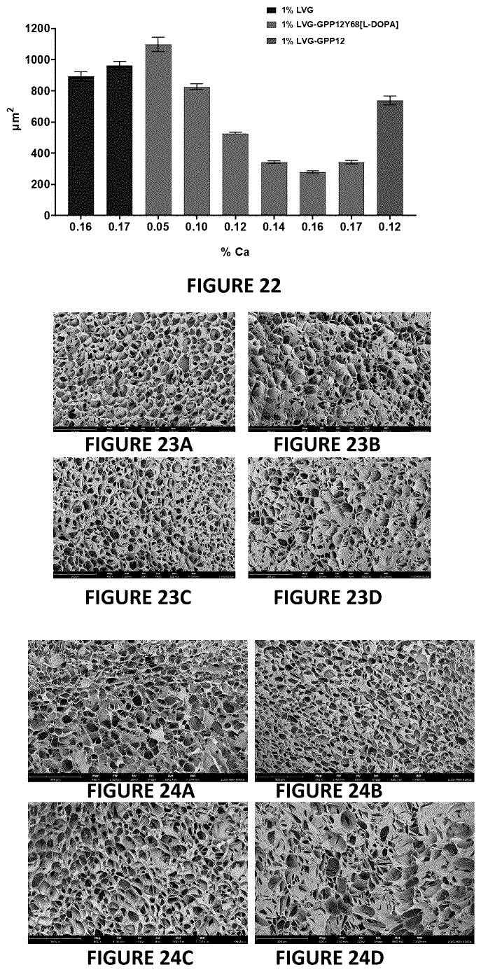

Next, sponge scaffolds were fabricated using the freeze-drying technique. The sponges were composed of varying compositions, including 1% LVG, 1% LVG-GPP12Y68[L-DOPA], and 1% LVG-GPP12 cross-linked with 0.05-0.17% calcium. Analysis via scanning electron microscopy (SEM) revealed distinct pore structures among the different scaffold compositions (FIGS. 22-25).

The presence of L-DOPA within the current collagen-mimicking peptide results in a decrease in scaffold pore size compared to scaffolds formed with peptide lacking L-DOPA or in the complete absence of the peptide (FIG. 22).

At higher concentrations of calcium, which facilitate stronger cross-linking, the pores of LVG-GPP12Y68[L-DOPA] scaffolds are observed to be dramatically smaller compared to those of scaffolds composed of LVG alone (FIG. 23).

The inventors conducted experiments with lower cross-linking concentrations to augment the pore size of LVG-GPP12Y68[L-DOPA] sponges, and indeed, reducing the calcium concentration resulted in an increase in pore size (FIG. 24).

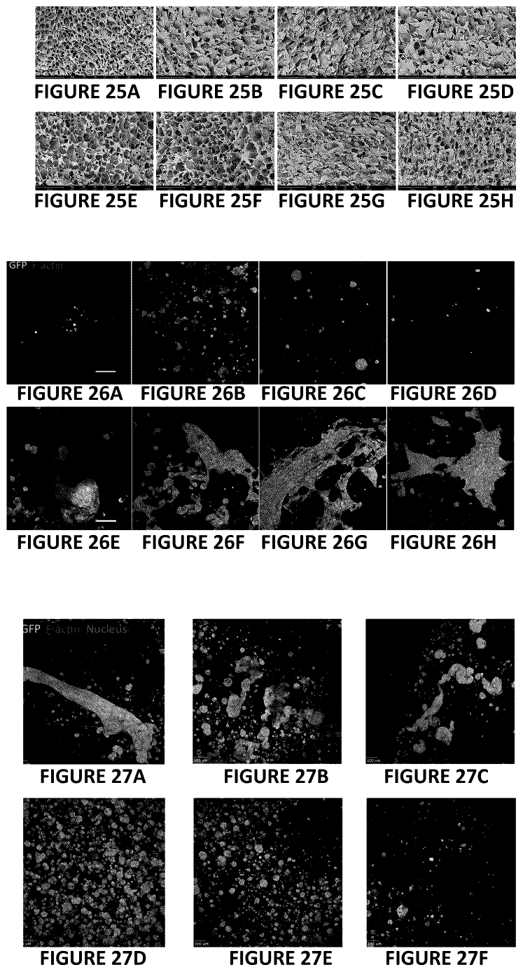

Ultimately, the inventors compared LVG coupled with collagen-mimicking peptide, with and without L-DOPA. In this comparison as well, the presence of L-DOPA was found to decrease the pore size (FIG. 25).

To investigate the potential of L-DOPA to induce cell spreading in a three-dimensional (3D) environment, the inventors cultured HFGFP cells on the current freeze-dried sponge scaffolds. These scaffolds were made of diverse compositions, including 1% LVG, 1% LVG-GPP12Y68[L-DOPA], and 1% LVG-GPP12, cross-linked with calcium concentrations ranging from 0.05% to 0.17%.

To address the larger pore size observed in LVG sponges, the inventors employed a high cross-linking calcium concentration of 0.16%. This facilitated a comparison of cell spreading between LVG and LVG-GPP12Y68[L-DOPA] sponges. Remarkably, for 14 days culture, cell spreading was only evident in the presence of the current L-DOPA collagen-mimicking peptide (FIG. 26).

The inventors utilized a lower calcium concentration of 0.12% to evaluate cell spreading and viability on scaffolds comprising LVG-GPP12 and LVG-GPP12Y68[L-DOPA]. The inventors examine whether cell spreading is attributed to L-DOPA. Consistent with the current comparison with LVG alone, it was evident that cell spreading occurred solely in the presence of GPP12Y68[L-DOPA] during the cell culture period (FIG. 27).

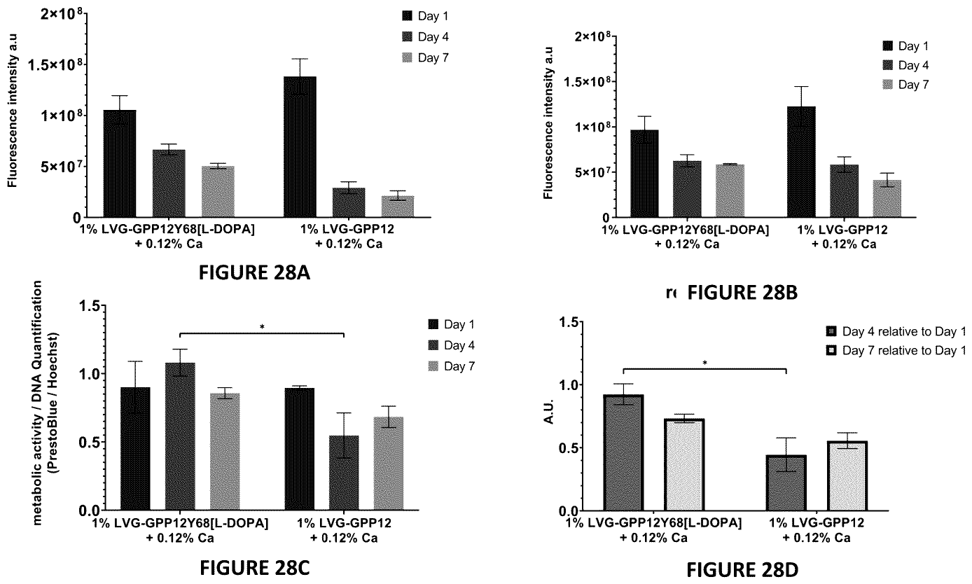

The inventors found that the presence of L-DOPA not only influences cell spreading but also affects cell viability (FIG. 28). This is significant because fibroblast cells require adhesion and spreading in order to properly function and proliferate effectively.

The inventors monitored metabolic activity using the PrestoBlue assay and quantified DNA levels using the Hoechst assay. The results show that in the absence of L-DOPA, a pronounced decrease in metabolic activity and cell quantity (as indicated by DNA quantification) was observed. decrease was more pronounced.

Upon examination of the normalized viability data, it became apparent that the presence of L-DOPA led to an increase in cell viability after 4 days of incubation (FIGS. 28C-28D). A notable and statistically significant difference in viability was observed between samples with or without L-DOPA, with the latter showing a decrease in cell viability in the current collagen-mimicking peptide.

### Example 9

### Enzymatic Synthetic of Collagen L-DOPA Polypeptides

To further increase incorporation efficiency of L-DOPA into the collagen mimicking polypeptides of the invention which were obtained by the orthogonal translation methodology (Example 3), the inventors utilized the tyrosinase enzyme. The enzyme was employed during peptide purification through affinity chromatography on Chitin resin. Through this method, the inventors successfully produced a peptide sample including 28.7% GPP12 with one (1) L-DOPA substitution and 14.0% GPP12 with two (2) L-DOPA substitutions (Table 3).

TABLE 3
Mass spectra of GPP12 after on-column tyrosinase treatment. Presented
are the expected and deconvoluted mass of each peptide.

| | Expected | Experimental | | |
|---|---|---|---|---|
| | molecular | molecular | Fractional | |
| Peptide | weight [da] | weight [da] | abundance | Fit |
| GPP12 | 6,115.59 | 6,111.75 | 57.3% | GPP without the 1^{st} |
| | | | | Met |
| GPP12 + | 6,132.60 | 6,133.75 | 17.8% | GPP without the 1^{st} |
| [L-DOPA] | | | | Met with a single |
| | | | | L-DOPA |
| | | | | substitution |
| GPP12 + | 6,166.61 | 6,165.68 | 10.9% | GPP without the 1^{st} |
| [L- | | | | Met with a single |
| DOPA] + | | | | L-DOPA |
| S | | | | substitution |
| | | | | that forms a bond |
| | | | | with a sulfur atom |
| GPP12 + | 6,149.61 | 6,149.71 | 14.0% | GPP without the 1^{st} |
| 2[L- | | | | Met with a double |
| DOPA] | | | | L-DOPA |
| | | | | substitutions |

In view of the above, the inventors conclude that synthesis of collagen mimicking polypeptides comprising L-DOPA (substitution), may be achieved by numerous methods and tools, such as, but not limited to, enzymatic reactions, as well as orthogonal synthesis, as exemplified herein. Further, collagen mimicking polypeptides of the invention may include at least one L-DOPA substitution, e.g., C-terminally to the GPP repeat, or two L-DOP substitutions, e.g., one N-terminally to the GPP repeat and one C-terminally to the GPP repeat.

While the present invention has been particularly described, people skilled in the art will appreciate that many variations and modifications can be made. Therefore, the invention is not to be construed as restricted to the particularly described embodiments, and the scope and concept of the invention will be more readily understood by reference to the claims which follow.

1. A polypeptide comprising at least 80% homology to an amino acid sequence comprising: MGSGYGSGSWCGTTPGSWCGT-[GPP]ₙ-GRDGSPGGSGYGSG, wherein n is the number of repetitions of the [GPP] repeat, and wherein at least one amino acid residue located N-terminally to the [GPP]ₙ repeat, C-terminally to the [GPP]ₙ repeat, or both, is substituted by 1-3,4-dihydroxyphenylalanine (L-DOPA) or a functional analog thereof.

2. The polypeptide of claim 1, wherein said at least one amino acid residue located N-terminally to the [GPP]ₙ repeat, C-terminally to the [GPP]ₙ repeat, or both is a tyrosine residue.

3. The polypeptide of claim 1 or 2, comprising the amino acid sequence: MGSGX₁GSGSWCGTTPGSWCGT-[GPP]n-GRDGSPGGSGX₂GSG, wherein n is the number of repetitions of the [GPP] repeat, and wherein any one of X₁ and X₂ is Tyrosine or L-DOPA, and optionally wherein: (i) when X₁ is Tyrosine X₂ is L-DOPA, and vice versa; or (ii) both X₁ and X₂ are L-DOPA.

4. (canceled)

5. The polypeptide of claim 1, wherein said n represents an integer ranging from 1 to 50.

6. The polypeptide of claim 1, wherein said n equals 12.

7. The polypeptide of claim 1, comprising the amino acid sequence: MGSGX₁GSGSWCGTTPGSWCGTGPPGPPGPPGPPGPPGPPGPPGPPGPPGPPGPPG PPGRDGSPGGSGX₂GSG (SEQ ID NO: 1), wherein: (i) when X₁ is Tyrosine X₂ is L-DOPA, or vice versa; or (ii) both X₁ and X₂ are L-DOPA, and optionally wherein said polypeptide comprises the amino acid sequence:
(SEQ ID NO: 4)
MGSGX₁GSGSWCGTTPGSWCGTGPPGPPGPPGPPGPPGPPGPPGPPGPP
GPPGPPGPPGRDGSPGGSGX₂GSG,
wherein X₁ and X₂ are L-DOPA.

8. (canceled)

9. The polypeptide of claim 1, in the form of a fiber, and optionally wherein: (i) said fiber is characterized by a length ranging from 5 nm to 1.5 μm; (ii) said fiber is characterized by a diameter ranging from 0.1 nm to 10 μm; or (iii) both (i) and (ii).

10.-11. (canceled)

12. The polypeptide of claim 1, characterized by any one of: (i) a porosity ranging from 30% to 90%; (ii) pores with an average size ranging from 1 μm to 200 μm; (iii) a tensile strength ranging from 10 MPa to 500 MPa; (iv) a young's modulus ranging from 1 KPa to 25 Kpa; or (v) any combination of (i) to (iv).

13.-15. (canceled)

16. The polypeptide of claim 1, further comprising a cross-linking modification, and optionally wherein said polypeptide is characterized by young's modulus ranging from 5 KPa to 20 KPa.

17. (canceled)

18. A polynucleotide comprising a nucleic acid sequence encoding the polypeptide of claim 1, and optionally wherein said polynucleotide comprises the acid nucleic sequence:
(SEQ ID NO: 2)
ATGGGTAGCGGTTATGGCAGCGGTAGCTGGTGCGGCACCACCCCGGGCA
GCTGGTGCGGTACCGGTCCGCCGGGTCCGCCGGGTCCGCCGGGCCCACC
GGGTCCGCCGGGTCCGCCAGGTCCACCGGGCCCGCCGGGCCCGCCAGGT
CCGCCGGGTCCACCGGGTCCGCCGGGTCGTGATGGTAGCCCGGGTGGTA
GTGGATAGGGAAGTGGA.

19. (canceled)

20. An artificial vector comprising the polynucleotide of claim 18, and optionally wherein said artificial vector is an expression vector or a plasmid.

21. (canceled)

22. A cell comprising the polynucleotide of claim 18, optionally wherein said cell is a prokaryote cell, and optionally wherein said cell is an Escherichia coli cell.

23.-24. (canceled)

25. A composition comprising the polypeptide of claim 1, and
an acceptable carrier, and optionally wherein: (i) said composition is in the form of: a sponge, a hydrogel, a hollow fiber tubing, a film, an ink, a patch, or a membrane; (ii) said composition is a porous composition, and optionally wherein said porous composition comprises pores with an average size ranging between 50 μm to 500 μm; or (iii) both (i) and (ii).

26.-29. (canceled)

30. A method for producing the polypeptide of claim 1, comprising the steps:
(a) providing a transformed bacterial cell comprising an artificial vector comprising a polynucleotide comprising a nucleic acid sequence encoding the polypeptide of claim 1; and
(b) culturing said transformed cell from step (a), wherein said culturing comprises contacting said transformed bacterial cell with a sufficient amount of L-DOPA,
thereby producing the polypeptide of claim 1.

31. The method of claim 30, further comprising: (i) a step preceding step (a), comprising transforming or transfecting said artificial vector into a bacterial cell, thereby providing said transformed bacterial cell; (ii) a step (c), comprising isolating, purifying, or both, said produced polypeptide; or (iii) both (i) and (ii).

32. (canceled)

33. The method of claim 30, wherein said transformed cell further comprises a transfer RNA (tRNA) synthase capable of ligating L-DOPA to a tRNA molecule, and optionally wherein said tRNA synthase is a modified tyrosyl-tRNA synthase.

34. (canceled)

35. The method of claim 33, wherein an anti-codon of said tRNA molecule comprises a nucleic acid sequence of CUA.

36. The method of claim 33, further comprising a step preceding step (a) comprising transforming or transfecting said bacterial cell with a polynucleotide comprising any one of: a nucleic acid sequence of said tRNA molecule, a nucleic acid sequence encoding said tRNA synthase, and both, optionally wherein said polynucleotide comprises a plurality of polynucleotide types, and optionally wherein a first type of said plurality of polynucleotide types comprises a nucleic acid sequence of said tRNA molecule, and wherein a second type of said plurality of polynucleotide types encodes said tRNA synthase.

37.-38. (canceled)

39. A method for producing a polypeptide comprising at least 80% homology to an amino acid sequence comprising MGSGX₁GSGSWCGTTPGSWCGT-[GPP]ₙ-GRDGSPGGSGX₂GSG, wherein n is the number of repetitions of the [GPP] repeat, the method comprising contacting a polypeptide comprising at least 80% homology to the amino acid sequence: MGSGYGSGSWCGTTPGSWCGT-[GPP]ₙ-GRDGSPGGSGYGSG with an effective amount of a tyrosinase enzyme or a functional analog thereof, wherein any one of X1 and X2 is Tyrosine or L-DOPA, and: (i) when X₁ is Tyrosine X₂ is L-DOPA, and vice versa; or (ii) both X₁ and X₂ are L-DOPA.

40. A composition comprising a plurality of molecules of said polypeptide produced according to the method of claim 30, and an acceptable carrier, optionally wherein at least 80% of said plurality of molecules of said polypeptide comprise a L-DOPA or at least 95% of said plurality of molecules of said polypeptide comprise a L-DOPA.

41.-42. (canceled)


SEQUENCE LISTING (4 sequences)

SEQ ID NO: 1 - protein, 71 aa, Synthetic construct
  VAR_SEQ 68
  VAR_SEQ 5
MGSGXGSGSWCGTTPGSWCGTGPPGPPGPPGPPGPPGPPGPPGPPGPPGPPGPPGPPGRDGSPGGSGXGSG

SEQ ID NO: 2 - DNA, 213 bp, Synthetic construct
  source 1..213: mol_type = other DNA
atgggtagcggttatggcagcggtagctggtgcggcaccaccccgggcagctggtgcggtaccggtccgccgggtccgccgggtccgccgggcccaccgggtccgccgggtccgccaggtccaccgggcccgccgggcccgccaggtccgccgggtccaccgggtccgccgggtcgtgatggtagcccgggtggtagtggatagggaagtgga

SEQ ID NO: 3 - DNA, 558 bp, Synthetic construct
  source 1..558: mol_type = other DNA
atgggtggtagcggttatggcagcggtagctggtgcggcaccaccccgggcagctggtgcggtaccggtccgccgggtccgccgggtccgccgggcccaccgggtccgccgggtccgccaggtccaccgggcccgccgggcccgccaggtccgccgggtccaccgggtccgccgggcccaccgggtccaccgggtccgccaggtccgccaggcccgccgggtccaccaggcccgccaggcccaccgggcccaccaggcccgccaggtccaccaggtccaccaggcccaccaggtccgccgggcccaccgggtccgccaggcccgccaggtccgccaggcccaccgggtccaccgggcccgccaggcccgccgggtccgccaggcccaccgggcccgccgggtccaccgggtccaccaggtccgccgggtccaccaggtccgccgggcccgccaggtccaccgggcccaccaggtccgccgggtccgccgggtccgccgggtccgccgggtccgccgggtcgtgatggtagcccgggtggtagtggatagggaagtgga

SEQ ID NO: 4 - protein, 71 aa, Synthetic construct; identical to SEQ ID NO: 1
  VAR_SEQ 5
  VAR_SEQ 68